(19) United States
(12) Patent Application Publication
(10) Pub. No.: US 2026/0192015 A1
(43) Pub. Date: Jul. 9, 2026

(54) HYDROGEN SULFIDE-RELEASING GRAFTS

(71) Applicant: MICHIGAN TECHNOLOGICAL UNIVERSITY, Houghton, MI (US)

(72) Inventor: Xiaochu Ding, Houghton, MI (US)

(21) Appl. No.: 19/015,174

(22) Filed: Jan. 9, 2025

(51) Int. Cl. A61L 27/18; A61L 27/54; A61L 27/58; C08L 67/04; D01F 6/62; D06M 10/00; D06M 101/32

(52) U.S. Cl. CPC A61L 27/18; A61L 27/54; A61L 27/58; C08L 67/04; D01F 6/625; D06M 10/001; A61L 2300/10; A61L 2430/40; C08L 2201/06; C08L 2312/06; D06M 2101/32; D10B 2401/061

(57) ABSTRACT

Exemplary hydrogen sulfide-releasing grafts may comprise backbones formed of first polymers and second polymers. Exemplary grafts may be biocompatible and degradable. Various methods may be used to generate exemplary grafts. Certain methods may involve electrospinning and, in some instances, the application of UV-light.

### GOVERNMENT INTEREST

This invention was made with government support under contract number R15HL159602 awarded by the National Institutes of Health. The government has certain rights in the invention.

### TECHNICAL FIELD

The present disclosure relates to material, methods, and techniques related to hydrogen sulfide-releasing grafts. Exemplary grafts may be resorbable.

### INTRODUCTION

Cardiovascular diseases (CVD), including occlusive artery diseases, are the leading cause of death worldwide. In the U.S. alone, there are about 400,000 coronary bypasses and 460,000 peripheral artery bypasses performed each year. Currently, autografts (e.g., saphenous vein and internal mammary artery) are the gold standard. However, approximately a third of these patients need synthetic grafts for treatment due to a shortage of the autologous resources. Nondegradable grafts such as poly(ethylene terephthalate) and expanded poly(tetrafluoroethylene) have been alternatives to the autografts. However, they perform poorly in small-diameter arteries (<6 mm) because of thrombosis, stenosis, and low patency rate. Therefore, bioresorbable grafts have been proposed for in situ arterial regeneration. Using the host regenerative capacities, the resorbable graft can be transformed into a living conduit with a structure and performance like a native artery as the polymer is gradually degraded and absorbed by host. This feature is particularly important for pediatric patients.

A polymer-based resorbable graft lacks bioactivity, and will likely induce complications such as inflammation, thrombosis, intimal hyperplasia, and calcification during the remodeling process. To inhibit these complications and improve the remodeling outcomes, scientists have explored strategies by functionalizing the resorbable grafts with active biomolecules. For example, growth factors and cell adhesion peptides have been used to functionalize the grafts to promote endothelialization and improve patency rate. Platelet-rich plasma-derived proteins were also used to promote endothelialization, smooth muscle cell infiltration and proliferation, and extracellular matrix deposition. Heparin has been widely used as an anti-coagulant to inhibit thrombosis and improve the patency rate. Each of these active biomolecules plays relatively simple roles in regulating the graft remodeling.

### SUMMARY

In some aspects, the techniques described herein relate to an elastomeric graft including: a backbone including a first polymer crosslinked with a second polymer, the first polymer including:

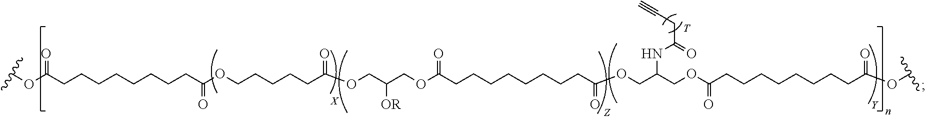

and where a ratio of X to (Y+Z) is between 20:80 and 55:45; where a ratio of Y to Z is between 10:90 and 25:75; where T is an integer from 0 to 6; where R is hydrogen or a polyester chain; where n is an integer from 2 to 50; and a second polymer including:

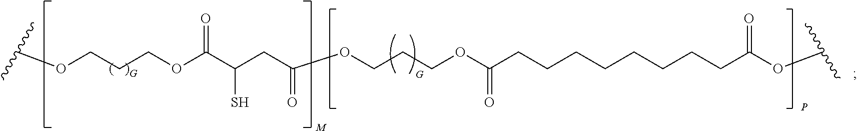

where a ratio of M to P is between 40:60 and 60:40; and where G is an integer from 1 to 6.

In some aspects, the techniques described herein relate to a method for preparing an elastomeric graft, the method including: combining a compound of formula (I) with glycerol, sebacic acid, and a solvent in a first vessel, where formula (I) is:

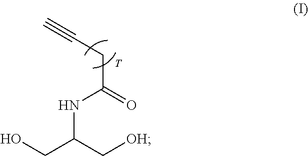

and where T is an integer from 0 to 6; and heating the first vessel to a temperature of 100° C. to 150° C.; while heating, purging the first vessel with nitrogen (N2) for a time period of 20 hours to 28 hours; after purging with nitrogen and while heating, applying a vacuum to the first vessel for a time period of 3 hours to 7 hours; thereby generating a compound of formula (II):

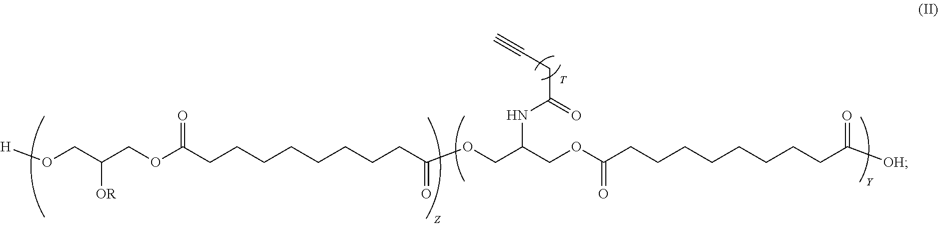

and where a ratio of Y to Z is between 10:90 and 25:75; and where R is hydrogen or a polyester chain; combining the compound of formula (II) with poly(ε-caprolactone), sebacic acid, and a second solvent in a second vessel; heating the second vessel to a temperature of 100° C. to 150° C.; while heating, purging the second vessel with nitrogen for a time period of 20 hours to 28 hours; after purging with nitrogen and while heating, applying a vacuum to the second vessel for a time period of 7 hours to 40 hours; thereby generating a first polymer; combining a compound of formula (III), sebacic acid, a compound of formula (IV) and a third solvent in a third vessel, where formula (III) is:

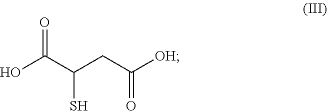

and where formula (IV) is:

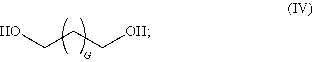

and where G is an integer from 1 to 6; heating the third vessel to a temperature of 100° C. to 150° C.; while heating, purging the third vessel with nitrogen for a time period of 20 hours to 28 hours; after purging with nitrogen and while heating, applying a vacuum to the third vessel for a time period of 70 hours to 80 hours; thereby generating a second polymer; combining the first polymer, the second polymer, a fourth solvent, and a radical initiator to form a crosslinking mixture; and electrospinning the crosslinking mixture to generate graft fiber.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

### BRIEF DESCRIPTION OF THE DRAWINGS

FIG. 1 is a schematic that illustrates (A) the synthesis of an exemplary first polymer, PCL₅₀-b-PGSA₂₀, and (B) the synthesis of an exemplary second polymer, POST₅₀.

FIG. 2 is a schematic depicting gel permeation chromatography of (A) PCL₅₀-b-PGSA₂₀ and (C) POST₅₀ as well as (B) PCL₅₀-b-PGSA₂₀ from an initial synthetic attempt using a variation of a synthesis protocol. Light scattering (LS), refractive index (RI), and ultraviolet (UV) chromatograms are overlaid.

FIG. 3 is an image of a 1H NMR spectrum for a thioamide linker. Integration area ratio of the protons (Hₐ:H_{b}:H_{c}:H_{d}=2:3:4:1.8) was close to its theoretical ratio of 2:3:4:2.

FIG. 4 shows (A) the process of forming an electrospun and UV-crosslinked graft, (B) a representative photograph of a PAPT graft with 1-mm inner diameter using low molecular weight PA and PT polymers (Table 1), and (C) a representative photograph of a PAPT graft made from high molecular weight PA and PT polymers (Table 1, c).

FIG. 5 shows SEM images of electrospun grafts made from high molecular weight PA and PT polymers (PAPT_H): (A) PAPT_H at 30 wt./v %, Outer Surface; (B) PAPT_H at 30 wt./v %, Inner Surface; (C) PAPT_H at 30 wt./v %, Cross-Section; (D) PAPT_H at 32 wt./v %; Outer Surface; (E) PAPT_H at 32 wt./v %; Inner Surface; (F) PAPT_H at 32 wt./v %; Cross-Section. Scale bar: (A, B, D, E) 50 μm; (C) 300 μm. (F) 400 μm. L: lumen.

FIG. 6 shows SEM images of the electrospun graft made from low molecular weight PA and PT polymers at 29 wt./v % with different PA-to-PT weight ratios: (A) Outer Surface PAPT_L at 1:1; (B) Outer Surface PAPT_L at 1.2:1; (C) Outer surface PAPT_L at 1.4:1; (D) Inner Surface PAPT_L at 1:1; (E); Inner Surface PAPT_L at 1.2:1; (F) Inner Surface PAPT_L at 1.4:1; (G) Cross-Section PAPT_L at 1:1; (H) Cross-Section PAPT_L at 1.2:1; (I) Cross-Section PAPT_L at 1.4:1. Scale bar: (A-F) 50 μm, (G, H, I) 100 μm.

FIG. 7 shows (A) a process to make a thioamide-conjugated PAPT graft using a thermal initiator ACVA (H₂S-PAPT__{T}); (B) a representative photograph of a H₂S-PAPT__{T} graft; and SEM images of the H₂S-PAPT__{T} graft's outer surface (C), inner surface (D), and cross-section (E). Scale bar: (C) 50 μm, (E) 100 μm. L: lumen.

FIG. 8 is a figure showing (A) a process to make a thioamide-conjugated PAPT graft using a UV initiator (H₂S-PAPT__{UV}); (B) a representative photograph of a H₂S-PAPT__{UV} graft with a slightly yellowish appearance; and SEM images of the H₂S-PAPT__{UV} graft's outer surface (C), inner surface (D), and cross-section (E). SEM images of a control PAPT graft's outer surface (F), inner surface (G), and cross-section (H). Scale bar: (C, D, F, G) 50 μm, (E) 200 μm. (H) 100 μm. L: lumen.

FIG. 9 shows (A) a process to make a thioamide-conjugated PAPT graft using an electrospun mixture of thioamide linker, UV initiator, PA polymer, and PT polymer that was UV-cured, H₂S-PAPT__{E}, as well as SEM images of the graft's Outer Surface (B), Inner Surface (C), and Cross-Section (D). Scale bar: (B, C) 50 μm, (D) 100 μm. L: lumen.

FIG. 10 is a figure of graphs showing in vitro release test of three types of H₂S-PAPT grafts in pH 7.4 deionized water at 37° C. for 15 days: (A) H₂S-PAPT__{T}; (B) H₂S-PAPT__{UV}; and (C) H₂S-PAPT__{E}. (D) A representative standard curve of the methylene blue assay. H₂S-PAPT__{E} was sterilized by ethylene oxide for in vitro release test. Data represent mean value±SD (n=3).

FIG. 11 is a figure of graphs showing mechanical properties of PAPT grafts electrospun from high molecular PA and PT polymers (PAPT_H) and low molecular weight PA and PT polymers (PAPT_L). (A) Representative stress-strain curves. (B) Fracture strain (%). (C) Elastic modulus (E, MPa). ***p<0.0001. (D) Ultimate tensile strength (UTS, MPa). ***, p<0.0001. p value<0.05 is considered significantly different. Data represent mean value±SD (n=6).

FIG. 12 is a figure of graphs showing uniaxial tensile test of a control PAPT graft and three types of the H₂S-PAPT grafts. (A) Representative stress-strain curves. (B) Fracture strain, %. (C) Elastic modulus (E, MPa), ***p<0.0001. (D) Ultimate tensile strength (UTS, MPa). p value<0.05 is considered significantly different. Data represent mean value±SD (n=6).

FIG. 13 is a figure of representative photographs (A) H₂S-PAPT and PAPT control grafts, and (B) H₂S-PAPT and PAPT control grafts that were implanted in rat abdominal aorta interposition models for 5 months.

FIG. 14 is a figure of images showing staining of H₂S-PAPT and PAPT control grafts (A) H&E staining, and (B) Alizarin staining to detect calcium deposition. Scale bar: (A) 200 μm (left), 50 μm (right); (B) 200 μm (left), 100 μm (right).

FIG. 15 is a figure of images showing immunohistochemical staining of H₂S-PAPT and PAPT control grafts: (A) H₂S-PAPT, vWF for endothelial cells; (B) H₂S-PAPT, SMA for smooth muscle cells; (C) H₂S-PAPT, CD206 for M2 macrophages; (D) PAPT control, vWF for endothelial cells; (E) PAPT control, SMA for smooth muscle cells; and (F) PAPT control, CD206 for M2 macrophages. Scale bar: 100 μm.

### DETAILED DESCRIPTION

Exemplary hydrogen sulfide-releasing grafts may comprise backbones formed of first polymers and second polymers. Exemplary grafts may be biocompatible and degradable. Various methods may be used to generate exemplary grafts. Certain methods may involve electrospinning and, in some instances, the application of UV-light.

### I. Definitions

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifiers “about” or “approximately” used in connection with a quantity are inclusive of the stated value and has the meaning dictated by the context (for example, it includes at least the degree of error associated with the measurement of the quantity). These modifiers should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are contemplated. For another example, when a pressure range is described as being between ambient pressure and another pressure, a pressure that is ambient pressure is expressly contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 104th Ed., inside cover, and specific functional groups are defined as described therein.

### II. Exemplary Materials

Exemplary materials may include first polymers and second polymers that may be crosslinked to form exemplary backbones. Exemplary backbones may have thiol substituents, hydroxyl substituents, pendant alkynes, and pendant thioamides.

### A. Exemplary Backbones

Exemplary backbones typically comprise crosslinked alkyl polyesters that may be optionally substituted. Exemplary backbones may be first polymers and second polymers that are crosslinked. Exemplary crosslinking of first polymers with second polymers may be via vinyl thioether bonds.

Exemplary backbones comprise pendant thioamides bonded through thioether bonds. Exemplary pendant thioamides may be as shown below:

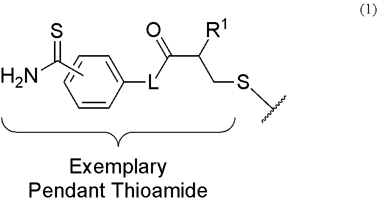

In the schematic above, exemplary pendant thioamides may comprise carbamothiophenyl propionate or carbamothiophenyl propionamide derivatives, where R¹ is hydrogen or methyl, and Lis O or NH.

Exemplary thioamides may be linked to exemplary backbones via aryl linkers. Linkers may be bonded to the carbon of the thioamide functional group of exemplary thioamides.

### B. Exemplary First Polymers

Exemplary first polymers typically comprise alkyl polyesters. Exemplary alkyl polyesters may be optionally substituted with hydroxyls and pendant alkyl alkynes.

Exemplary first polymers typically comprise varying repeat units connected by ester bonds, as shown below.

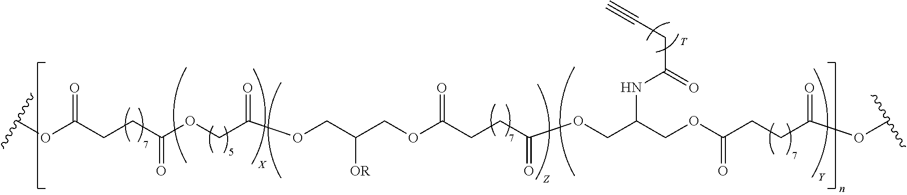

In the exemplary first polymer above, T is an integer that may be 0, 1, 2, 3, 4, 5, or 6.

In the exemplary first polymer, R is hydrogen or a polyester chain. A polyester chain may be created via crosslinked esterification of secondary alcohols with other polymer chains during polymer growth.

In the exemplary first polymer, n is an integer from 2 to 50.

Exemplary first polymers may have a Y to Z ratio between 10:90 and 25:75. In various instances, exemplary Y to Z ratios are between 10:90 and 25:75; 10:90 and 20:80; 10:90 and 15:85; 20:80 and 25:75; or 15:85 and 25:75.

Exemplary first polymers may have an X to (Y+Z) ratio between 20:80 and 55:45. In various instances, exemplary X to (Y+Z) ratios are between 20:80 and 55:45; 20:80 and 50:50; 20:80 and 45:55; 20:80 and 40:60; 20:80 and 35:65; 20:80 and 30:70; 20:80 and 25:75; 25:75 and 55:45; 30:70 and 55:45; 35:65 and 55:45; 40:60 and 55:45; 45:55 and 55:45; or 50:50 and 55:45.

Exemplary first polymers may have an average number molecular weight (Mn) between 1,800 Daltons and 47,500 Daltons. In various instances, exemplary first polymers have a Mn between 1,800 Daltons and 47,500 Daltons; 4,000 Daltons and 47,500 Daltons; 6,000 Daltons and 47,500 Daltons; 8,000 Daltons and 47,500 Daltons; 10,000 Daltons and 47,500 Daltons; 12,000 Daltons and 47,500 Daltons; 14,000 Daltons and 47,500 Daltons; 16,000 Daltons and 47,500 Daltons; 18,000 Daltons and 47,500 Daltons; 20,000 Daltons and 47,500 Daltons; 22,000 Daltons and 47,500 Daltons; 24,000 Daltons and 47,500 Daltons; 26,000 Daltons and 47,500 Daltons; 28,000 Daltons and 47,500 Daltons; 30,000 Daltons and 47,500 Daltons; 32,000 Daltons and 47,500 Daltons; 34,000 Daltons and 47,500 Daltons; 36,000 Daltons and 47,500 Daltons; 38,000 Daltons and 47,500 Daltons; 40,000 Daltons and 47,500 Daltons; 42,000 Daltons and 47,500 Daltons; 44,000 Daltons and 47,500 Daltons; 46,000 Daltons and 47,500 Daltons; 1,800 Daltons and 46,000 Daltons; 1,800 Daltons and 44,000 Daltons; 1,800 Daltons and 42,000 Daltons; 1,800 Daltons and 40,000 Daltons; 1,800 Daltons and 38,000 Daltons; 1,800 Daltons and 36,000 Daltons; 1,800 Daltons and 34,000 Daltons; 1,800 Daltons and 32,000 Daltons; 1,800 Daltons and 30,000 Daltons; 1,800 Daltons and 28,000 Daltons; 1,800 Daltons and 26,000 Daltons; 1,800 Daltons and 24,000 Daltons; 1,800 Daltons and 22,000 Daltons; 1,800 Daltons and 20,000 Daltons; 1,800 Daltons and 18,000 Daltons; 1,800 Daltons and 16,000 Daltons; 1,800 Daltons and 14,000 Daltons; 1,800 Daltons and 12,000 Daltons; 1,800 Daltons and 10,000 Daltons; 1,800 Daltons and 8,000 Daltons; 1,800 Daltons and 6,000 Daltons; or 1,800 Daltons and 4,000 Daltons. In various instances, first polymers may have a Mn no less than 1,800 Daltons; no less than 2,000 Daltons; no less than 4,000 Daltons; no less than 6,000 Daltons; no less than 8,000 Daltons; no less than 10,000 Daltons; no less than 12,000 Daltons; no less than 14,000 Daltons; no less than 16,000 Daltons; no less than 18,000 Daltons; no less than 20,000 Daltons; no less than 22,000 Daltons; no less than 24,000 Daltons; no less than 26,000 Daltons; no less than 28,000 Daltons; no less than 30,000 Daltons; no less than 32,000 Daltons; no less than 34,000 Daltons; no less than 36,000 Daltons; no less than 38,000 Daltons; no less than 40,000 Daltons; no less than 42,000 Daltons; no less than 44,000 Daltons; no less than 46,000 Daltons; or no less than 47,500 Daltons. In various instances, first polymers may have a Mn no greater than 47,500 Daltons; no greater than 46,000 Daltons; no greater than 44,000 Daltons; no greater than 42,000 Daltons; no greater than 40,000 Daltons; no greater than 38,000 Daltons; no greater than 36,000 Daltons; no greater than 34,000 Daltons; no greater than 32,000 Daltons; no greater than 30,000 Daltons; no greater than 28,000 Daltons; no greater than 26,000 Daltons; no greater than 24,000 Daltons; no greater than 22,000 Daltons; no greater than 20,000 Daltons; no greater than 18,000 Daltons; no greater than 16,000 Daltons; no greater than 14,000 Daltons; no greater than 12,000 Daltons; no greater than 10,000 Daltons; no greater than 8,000 Daltons; no greater than 6,000 Daltons; no greater than 4,000 Daltons; no greater than 2,000 Daltons; or no greater than 1,800 Daltons.

Exemplary first polymers may have a polydispersity index (PDI) between 5 and 68. In various instances, exemplary first polymers have a PDI between 5 and 68; 10 and 68; 15 and 68; 20 and 68; 25 and 68; 30 and 68; 35 and 68; 40 and 68; 45 and 68; 50 and 68; 55 and 68; 60 and 68; 65 and 68; 5 and 65; 5 and 60; 5 and 55; 5 and 50; 5 and 45; 5 and 40; 5 and 35; 5 and 30; 5 and 25; 5 and 20; 5 and 15; or 5 and 10. In various instances, exemplary first polymers may have a PDI no less than 5; no less than 10; no less than 15; no less than 20; no less than 25; no less than 30; no less than 35; no less than 40; no less than 45; no less than 50; no less than 55; no less than 60; no less than 65; or no less than 68. In various instances, first polymers may have a PDI no greater than 68; no greater than 65; no greater than 60; no greater than 55; no greater than 50; no greater than 45; no greater than 40; no greater than 35; no greater than 30; no greater than 25; no greater than 20; no greater than 15; no greater than 10; or no greater than 5.

### C. Exemplary Second Polymers

Exemplary second polymers may comprise an alkyl polyester optionally substituted with thiols.

Exemplary second polymers typically comprise varying repeat units connected by ester bonds, as shown below.

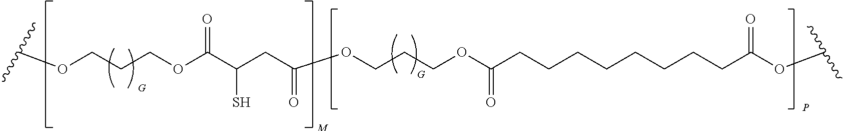

In an exemplary second polymer, G is an integer that may be 1, 2, 3, 4, 5, or 6.

Exemplary second polymers may have an M to P ratio between 40:60 and 60:40. In various instances, exemplary M to P ratios are between 40:60 and 60:40; 45:55 and 60:40; 50:50 and 60:40; 55:45 and 60:40; 40:60 and 55:45; 40:60 and 50:50; or 40:60 and 45:55.

Exemplary second polymers may have an average number molecular weight (Mn) between 6400 Daltons and 9600 Daltons. In various instances, exemplary Mn is between 6400 Daltons and 9600 Daltons; 6800 Daltons and 9600 Daltons; 7200 Daltons and 9600 Daltons; 7600 Daltons and 9600 Daltons; 8000 Daltons and 9600 Daltons; 8400 Daltons and 9600 Daltons; 8800 Daltons and 9600 Daltons; 9200 Daltons and 9600 Daltons; 6400 Daltons and 9200 Daltons; 6400 Daltons and 8800 Daltons; 6400 Daltons and 8400 Daltons; 6400 Daltons and 8000 Daltons; 6400 Daltons and 7600 Daltons; 6400 Daltons and 7200 Daltons; 6400 Daltons and 6800 Daltons; 7180 Daltons and 8990 Daltons; 7380 Daltons and 8990 Daltons; 7580 Daltons and 8990 Daltons; 7780 Daltons and 8990 Daltons; 7980 Daltons and 8990 Daltons; 8180 Daltons and 8990 Daltons; 8380 Daltons and 8990 Daltons; 8580 Daltons and 8990 Daltons; 8780 Daltons and 8990 Daltons; 7180 Daltons and 8790 Daltons; 7180 Daltons and 8590 Daltons; 7180 Daltons and 8390 Daltons; 7180 Daltons and 8190 Daltons; 7180 Daltons and 7990 Daltons; 7180 Daltons and 7790 Daltons; 7180 Daltons and 7590 Daltons; or 7180 Daltons and 7390 Daltons. In various instances, second polymers may have a Mn no less than 6400 Daltons; no less than 6800 Daltons; no less than 7180 Daltons; no less than 7600 Daltons; no less than 8000 Daltons; no less than 8400 Daltons; no less than 8800 Daltons; no less than 8990 Daltons; no less than 9200 Daltons; or no less than 9600 Daltons. In various instances, second polymers may have a Mn no greater than 9600 Daltons; no greater than 9200 Daltons; no greater than 8990 Daltons; no greater than 8800 Daltons; no greater than 8400 Daltons; no greater than 8000 Daltons; no greater than 7600 Daltons; no greater than 7200 Daltons; no greater than 7180 Daltons; no greater than 6800 Daltons; or no greater than 6400 Daltons.

Exemplary second polymers may have a polydispersity index (PDI) between 3 and 26. In various instances, exemplary PDI is between 3 and 26; 5 and 26; 7 and 26; 9 and 26; 11 and 26; 13 and 26; 15 and 26; 17 and 26; 19 and 26; 21 and 26; 23 and 26; 25 and 26; 3 and 25; 3 and 23; 3 and 21; 3 and 19; 3 and 17; 3 and 15; 3 and 13; 3 and 11; 3 and 9; 3 and 7; or 3 and 5. In various instances, second polymers may have a PDI no less than 3; no less than 5; no less than 7; no less than 9; no less than 11; no less than 13; no less than 15; no less than 17; no less than 19; no less than 21; no less than 23; no less than 25; or no less than 26. In various instances, second polymers may have a PDI no greater than 26; no greater than 25; no greater than 23; no greater than 21; no greater than 19; no greater than 17; no greater than 15; no greater than 13; no greater than 11; no greater than 9; no greater than 7; no greater than 5; or no greater than 3.

### III. Methods of Manufacture

Exemplary methods of manufacture may comprise making a compound of exemplary formula (II), forming a first polymer, forming a second polymer, electrospinning polymers, forming grafts and modifying grafts.

### A. Method of Making a Compound of Exemplary Formula (II)

An exemplary method of making a compound of formula (II), shown below, may include combining a compound of formula (I) with glycerol, sebacic acid, and a solvent in a vessel. In some instances, the solvent may be acetone.

Formula (I) may be:

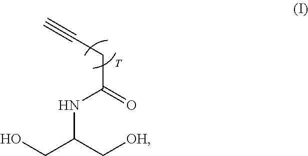

where T may be an integer from 0 to 6.

The example method may comprise heating the vessel to a temperature of 100° C. to 150° C. In various instances, the temperature may be between 100° C. and 150° C.; 100° C. and 140° C.; 100° C. and 130° C.; 100° C. and 120° C.; 100° C. and 110° C.; 110° C. and 150° C.; 120° C. and 150° C.; 130° C. and 150° C.; or 140° C. and 150° C. In various instances, the temperature may be no less than 100° C.; no less than 110° C.; no less than 120° C.; no less than 130° C.; no less than 140° C.; or no less than 150° C. In various instances, the temperature may be no greater than 150° C.; no greater than 140° C.; no greater than 130° C.; no greater than 120° C.; no greater than 110° C.; or no greater than 100° C.

While heating, the example method may comprise purging the vessel with nitrogen or argon for a time period that may be 20 hours to 28 hours. In various instances, the time period may be between 20 hours and 28 hours; 21 hours and 28 hours; 22 hours and 28 hours; 23 hours and 28 hours; 24 hours and 28 hours; 25 hours and 28 hours; 26 hours and 28 hours; 27 hours and 28 hours; 20 hours and 27 hours; 20 hours and 26 hours; 20 hours and 25 hours; 20 hours and 24 hours; 20 hours and 23 hours; 20 hours and 22 hours; or 20 hours and 21 hours. In various instances, the time period may be no less than 20 hours; no less than 21 hours; no less than 22 hours; no less than 23 hours; no less than 24 hours; no less than 25 hours; no less than 26 hours; no less than 27 hours; or no less than 28 hours. In various instances, the time period may be no greater than 28 hours; no greater than 27 hours; no greater than 26 hours; no greater than 25 hours; no greater than 24 hours; no greater than 23 hours; no greater than 22 hours; no greater than 21 hours; or no greater than 20 hours.

After purging, and while heating, the example method may comprise applying a vacuum to the vessel for a time period that may be 3 hours to 7 hours. In various instances, the time period may be between 3 hours and 7 hours; 3 hours and 6 hours; 3 hours and 5 hours; 3 hours and 4 hours; 4 hours and 7 hours; 5 hours and 7 hours; or 6 hours and 7 hours. In various instances, the time period may be no less than 3 hours; no less than 4 hours; no less than 5 hours; no less than 6 hours; or no less than 7 hours. In various instances, the time period may be no greater than 7 hours; no greater than 6 hours; no greater than 5 hours; no greater than 4 hours; or no greater than 3 hours.

After heating and applying a vacuum, a compound of formula (II) may be formed, where the compound of formula (II) may be:

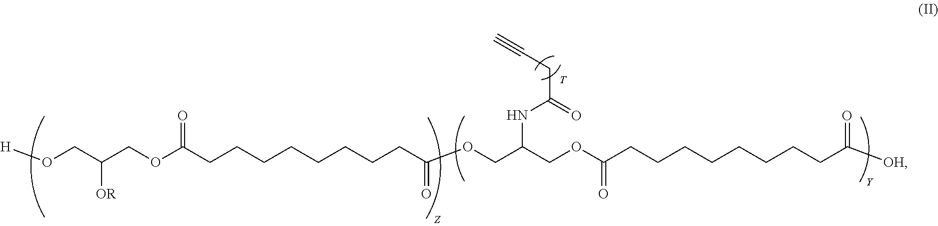

where a ratio of Y to Z may be between 10:90 and 25:75, and where R may be hydrogen or a polyester chain.

### B. Method of Making Exemplary First Polymers

An exemplary method of making a first polymer may include combining a compound of formula (II) with poly(ε-caprolactone), sebacic acid, and a solvent in a vessel. In some instances, the solvent may be 1,4-dioxane.

The example method may comprise heating the vessel to a temperature of 100° C. to 150° C. In various instances, the temperature may be between 100° C. and 150° C.; 100° C. and 140° C.; 100° C. and 130° C.; 100° C. and 120° C.; 100° C. and 110° C.; 110° C. and 150° C.; 120° C. and 150° C.; 130° C. and 150° C.; or 140° C. and 150° C. In various instances, the temperature may be no less than 100° C.; no less than 110° C.; no less than 120° C.; no less than 130° C.; no less than 140° C.; or no less than 150° C. In various instances, the temperature may be no greater than 150° C.; no greater than 140° C.; no greater than 130° C.; no greater than 120° C.; no greater than 110° C.; or no greater than 100° C.

While heating, the example method comprises stirring the reactants in the vessel under a nitrogen atmosphere for a time period that may be between 2 hours to 6 hours. In various instances, the example method comprises stirring the reactants in the vessel under a nitrogen atmosphere for a time period of between 2 hours and 6 hours; 3 hours and 6 hours; 4 hours and 6 hours; 5 hours and 6 hours; 2 hours and 5 hours; 2 hours and 4 hours; or 2 hours and 3 hours. In various instances, the example method comprises stirring the reactants in the vessel under a nitrogen atmosphere for a time period of no less than 2 hours; 3 hours; 4 hours; 5 hours; or 6 hours. In various instances, the example method comprises stirring the reactants in the vessel under a nitrogen atmosphere for a time period of no greater than 6 hours; 5 hours; 4 hours; 3 hours; or 2 hours.

While heating, the example method may comprise purging the vessel with nitrogen or argon for a time period that may be 20 hours to 28 hours. In various instances, the time period may be between 20 hours and 28 hours; 21 hours and 28 hours; 22 hours and 28 hours; 23 hours and 28 hours; 24 hours and 28 hours; 25 hours and 28 hours; 26 hours and 28 hours; 27 hours and 28 hours; 20 hours and 27 hours; 20 hours and 26 hours; 20 hours and 25 hours; 20 hours and 24 hours; 20 hours and 23 hours; 20 hours and 22 hours; or 20 hours and 21 hours. In various instances, the time period may be no less than 20 hours; no less than 21 hours; no less than 22 hours; no less than 23 hours; no less than 24 hours; no less than 25 hours; no less than 26 hours; no less than 27 hours; or no less than 28 hours. In various instances, the time period may be no greater than 28 hours; no greater than 27 hours; no greater than 26 hours; no greater than 25 hours; no greater than 24 hours; no greater than 23 hours; no greater than 22 hours; no greater than 21 hours; or no greater than 20 hours.

After purging, and while heating, the example method may comprise applying a vacuum to the vessel for a time period that may be 7 hours to 40 hours. In various instances, the time period may be between 7 hours and 40 hours; 10 hours and 40 hours; 13 hours and 40 hours; 16 hours and 40 hours; 19 hours and 40 hours; 22 hours and 40 hours; 25 hours and 40 hours; 28 hours and 40 hours; 31 hours and 40 hours; 34 hours and 40 hours; 37 hours and 40 hours; 7 hours and 37 hours; 7 hours and 34 hours; 7 hours and 31 hours; 7 hours and 28 hours; 7 hours and 25 hours; 7 hours and 22 hours; 7 hours and 19 hours; 7 hours and 16 hours; 7 hours and 13 hours; or 7 hours and 10 hours. In various instances, the time period may be no less than 7 hours; no less than 10 hours; no less than 13 hours; no less than 16 hours; no less than 19 hours; no less than 22 hours; no less than 25 hours; no less than 28 hours; no less than 31 hours; no less than 34 hours; no less than 37 hours; or no less than 40 hours. In various instances, the time period may be no greater than 40 hours; no greater than 37 hours; no greater than 34 hours; no greater than 31 hours; no greater than 28 hours; no greater than 25 hours; no greater than 22 hours; no greater than 19 hours; no greater than 16 hours; no greater than 13 hours; no greater than 10 hours; or no greater than 7 hours.

After heating and applying a vacuum, a first polymer may be formed.

### C. Method of Making Exemplary Second Polymers

An exemplary method of making a second polymer may include combining thiomalic acid, sebacic acid, a compound of formula (IV), and a solvent in a vessel. In some instances, the solvent may be acetone.

Formula (IV) may be

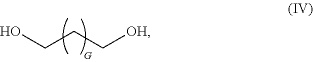

where G may be an integer from 1 to 6.

The example method may comprise heating the vessel to a temperature of 100° C. to 150° C. In various instances, the temperature may be between 100° C. and 150° C.; 100° C. and 140° C.; 100° C. and 130° C.; 100° C. and 120° C.; 100° C. and 110° C.; 110° C. and 150° C.; 120° C. and 150° C.; 130° C. and 150° C.; or 140° C. and 150° C. In various instances, the temperature may be no less than 100° C.; no less than 110° C.; no less than 120° C.; no less than 130° C.; no less than 140° C.; or no less than 150° C. In various instances, the temperature may be no greater than 150° C.; no greater than 140° C.; no greater than 130° C.; no greater than 120° C.; no greater than 110° C.; or no greater than 100° C.

While heating, the example method may comprise purging the vessel with nitrogen or argon for a time period that may be 20 hours to 28 hours. In various instances, the time period may be between 20 hours and 28 hours; 21 hours and 28 hours; 22 hours and 28 hours; 23 hours and 28 hours; 24 hours and 28 hours; 25 hours and 28 hours; 26 hours and 28 hours; 27 hours and 28 hours; 20 hours and 27 hours; 20 hours and 26 hours; 20 hours and 25 hours; 20 hours and 24 hours; 20 hours and 23 hours; 20 hours and 22 hours; or 20 hours and 21 hours. In various instances, the time period may be no less than 20 hours; no less than 21 hours; no less than 22 hours; no less than 23 hours; no less than 24 hours; no less than 25 hours; no less than 26 hours; no less than 27 hours; or no less than 28 hours. In various instances, the time period may be no greater than 28 hours; no greater than 27 hours; no greater than 26 hours; no greater than 25 hours; no greater than 24 hours; no greater than 23 hours; no greater than 22 hours; no greater than 21 hours; or no greater than 20 hours.

After purging, and while heating, the example method may comprise applying a vacuum to the vessel for a time period that may be 70 hours to 80 hours. In various instances, the time period may be between 70 hours and 80 hours; 71 hours and 80 hours; 72 hours and 80 hours; 73 hours and 80 hours; 74 hours and 80 hours; 75 hours and 80 hours; 76 hours and 80 hours; 77 hours and 80 hours; 78 hours and 80 hours; 79 hours and 80 hours; 70 hours and 79 hours; 70 hours and 78 hours; 70 hours and 77 hours; 70 hours and 76 hours; 70 hours and 75 hours; 70 hours and 74 hours; 70 hours and 73 hours; 70 hours and 72 hours; or 70 hours and 71 hours. In various instances, the time period may be no less than 70 hours; no less than 71 hours; no less than 72 hours; no less than 73 hours; no less than 74 hours; no less than 75 hours; no less than 76 hours; no less than 77 hours; no less than 78 hours; no less than 79 hours; or no less than 80 hours. In various instances, the time period may be no greater than 80 hours; no greater than 79 hours; no greater than 78 hours; no greater than 77 hours; no greater than 76 hours; no greater than 75 hours; no greater than 74 hours; no greater than 73 hours; no greater than 72 hours; no greater than 71 hours; or no greater than 70 hours.

After heating and applying a vacuum, a second polymer may be formed.

### D. Generating Exemplary Grafts

Exemplary methods may include generating grafts using first polymers and second polymers. Generating grafts may comprise electrospinning polymer solutions. In some instances, generating grafts may comprise applying UV light after electrospinning.

In some instances, generating grafts may comprise attaching pendant thioamide groups after electrospinning. Various aspects of each are discussed below.

Exemplary polymer solutions, alternatively referred to as crosslinking mixtures, used during electrospinning may comprise first polymer, second polymer, solvent, and a radical initiator. In some instances, exemplary polymer solutions used during electrospinning may additionally comprise a thioamide group linker.

In some instances, the polymer/solvent wt./v % may comprise values between 25 wt./v % and 35 wt./v %. In various instances, the wt./v % may be between 25 wt./v % and 35 wt./v %; 26 wt./v % and 35 wt./v %; 27 wt./v % and 35 wt./v %; 28 wt./v % and 35 wt./v %; 29 wt./v % and 35 wt./v %; 30 wt./v % and 35 wt./v %; 31 wt./v % and 35 wt./v %; 32 wt./v % and 35 wt./v %; 33 wt./v % and 35 wt./v %; 34 wt./v % and 35 wt./v %; 25 wt./v % and 34 wt./v %; 25 wt./v % and 33 wt./v %; 25 wt./v % and 32 wt./v %; 25 wt./v % and 31 wt./v %; 25 wt./v % and 30 wt./v %; 25 wt./v % and 29 wt./v %; 25 wt./v % and 28 wt./v %; 25 wt./v % and 27 wt./v %; or 25 wt./v % and 26 wt./v %. In various instances, the wt./v % may be no less than 25 wt./v %; no less than 26 wt./v %; no less than 27 wt./v %; no less than 28 wt./v %; no less than 29 wt./v %; no less than 30 wt./v %; no less than 31 wt./v %; no less than 32 wt./v %; no less than 33 wt./v %; no less than 34 wt./v %; or no less than 35 wt./v %. In various instances, the wt./v % may be no greater than 35 wt./v %; no greater than 34 wt./v %; no greater than 33 wt./v %; no greater than 32 wt./v %; no greater than 31 wt./v %; no greater than 30 wt./v %; no greater than 29 wt./v %; no greater than 28 wt./v %; no greater than 27 wt./v %; no greater than 26 wt./v %; or no greater than 25 wt./v %.

In some instances, the solvent may be acetone, dichloromethane, or a mixture thereof.

In some instances, the radical initiator may be irgacure 2959. In some instances, the radical initiator may be 2-hydroxy-4′-(2-hydroxyethoxy)-2-methylpropiophenone derivatives, azobisisobutyronitrile, azobisisobutyronitrile derivatives, benzoyl peroxide, benzoyl peroxide derivatives, 2,2-dimethoxy-2-phenylacetophenone, or 2,2-dimethoxy-2-phenylacetophenone derivatives.

An example method of electrospinning may comprise loading a plastic syringe with a polymer solution. The example method may comprise fitting a plastic syringe with a blunt needle. The example method may comprise placing the loaded syringe injection pump. The example method may comprise connecting the syringe needle to a high-voltage power supply. The example method may comprise using a grounded 1-mm stainless steel mandrel as a collector. The example method may comprise coating the mandrel with a layer of polyethylene oxide and drying it. The example method may comprise various electrospinning parameters such as an injection rate, a voltage, a distance between the needle tip and the mandrel, and a mandrel rotating speed of approximately 6.0 to 40.0 μl/min, 12.0 to 17.0 kV, 14 to 39 cm, and 30 to 100 rpm, respectively.

In various instances, the example method may comprise electrospinning with a mandrel diameter of 1 mm to 6 mm. In various instances, the example method may comprise electrospinning with a mandrel diameter between 1 mm and 6 mm; 2 mm and 6 mm; 3 mm and 6 mm; 4 mm and 6 mm; 5 mm and 6 mm; 1 mm and 5 mm; 1 mm and 4 mm; 1 mm and 3 mm; or 1 mm and 2 mm. In various instances, the example method may comprise electrospinning with a mandrel diameter no less than 1 mm; no less than 2 mm; no less than 3 mm; no less than 4 mm; no less than 5 mm; or no less than 6 mm. In various instances, the example method may comprise electrospinning with a mandrel diameter no greater than 6 mm; no greater than 5 mm; no greater than 4 mm; no greater than 3 mm; no greater than 2 mm; or no greater than 1 mm.

In various instances, the example method may comprise an electrospinning injection rate of 6.0 to 40.0 μl/min. In various instances, the example method comprises an electrospinning injection rate of 6.0 to 40.0 μl/min; 6.0 to 35.0 μl/min; 6.0 to 30.0 μl/min; 6.0 to 25.0 l/min; 6.0 to 20.0 μl/min; 6.0 to 15.0 μl/min; 6.0 to 10.0 μl/min; 10.0 to 40.0 μl/min; 15.0 to 40.0 μl/min; 20.0 to 40.0 μl/min; 25.0 to 30.0 μl/min; or 35.0 to 40.0 μl/min. In various instances, the example method may comprise an electrospinning injection rate of no less than 6 μl/min; no less than 10 μl/min; no less than 15 μl/min; no less than 20 μl/min; no less than 25 μl/min; no less than 30 μl/min; no less than 35 μl/min; or no less than 40 μl/min. In various instances, the example method may comprise an electrospinning injection rate of no greater than 40 μl/min; no greater than 35 μl/min; no greater than 30 μl/min; no greater than 25 μl/min; no greater than 20 μl/min; no greater than 15 μl/min; no greater than 10 μl/min; or no greater than 6 μl/min.

In various instances, the example method comprises an electrospinning voltage between 12.0 kV and 17.0 kV; 13.0 kV and 17.0 kV; 14.0 kV and 17.0 kV; 15.0 kV and 17.0 kV; 16.0 kV and 17.0 kV; 12.0 kV and 16.0 kV; 12.0 kV and 15.0 kV; 12.0 kV and 14.0 kV; or 12.0 kV and 13.0 kV. In various instances, the example method comprises an electrospinning voltage of no less than 12.0 kV; no less than 13.0 kV; no less than 14.0 kV; no less than 15.0 kV; no less than 16.0 kV; or no less than 17.0 kV. In various instances, the example method comprises an electrospinning voltage of no greater than 17.0 kV; no greater than 16.0 kV; no greater than 15.0 kV; no greater than 14.0 kV; no greater than 13.0 kV; or no greater than 12.0 kV.

In various instances, the example method comprises an electrospinning distance between the needle tip and the mandrel of between 14 and 39 cm; 20 and 39 cm; 25 and 39 cm; 30 and 39 cm; 35 and 39 cm; 14 and 35 cm; 14 and 30 cm; 14 and 25 cm; or 14 and 20 cm. In various instances, the example method comprises an electrospinning distance between the needle tip and the mandrel of no less than 14 cm; no less than 20 cm; no less than 25 cm; no less than 30 cm; no less than 35 cm; or no less than 39 cm. In various instances, the example method comprises an electrospinning distance between the needle tip and the mandrel of no greater than 39 cm; no greater than 35; no greater than 30; no greater than 25 cm; no greater than 20 cm; or no greater than 14 cm.

In various instances, the example method comprises an electrospinning mandrel rotating speed of between 30 rpm and 100 rpm; 40 rpm and 100 rpm; 50 rpm and 100 rpm; 60 rpm and 100 rpm; 70 rpm and 100 rpm; 80 rpm and 100 rpm; 90 rpm and 100 rpm; 30 rpm and 90 rpm; 30 rpm and 80 rpm; 30 rpm and 70 rpm; 30 rpm and 60 rpm; 30 rpm and 50 rpm; or 30 rpm and 40 rpm. In various instances, the example method comprises an electrospinning mandrel rotating speed of no less than 30 rpm; no less than 40 rpm; no less than 50 rpm; no less than 60 rpm; no less than 70 rpm; no less than 80 rpm; no less than 90 rpm; or no less than 100 rpm. In various instances, the example method comprises an electrospinning mandrel rotating speed of no greater than 100 rpm; no greater than 90 rpm; no greater than 80 rpm; no greater than 70 rpm; no greater than 60 rpm; no greater than 50 rpm; no greater than 40 rpm; or no greater than 30 rpm.

The example method may comprise collecting electrospun fibers on a rotating mandrel.

The example method may comprise exposing the electrospun fibers to UV light between 254 nm and 400 nm at approximately room temperature. In various instances, the UV wavelength may be between 254 nm and 400 nm; 254 nm and 400 nm; 275 nm and 400 nm; 300 nm and 400 nm; 325 nm and 400; 350 nm and 400 nm; 375 nm and 400; 254 nm and 375 nm; 254 nm and 350 nm; 254 nm and 325 nm; 254 nm and 300 nm; or 254 nm and 275 nm. In various instances, the UV wavelength may be no less than 254 nm; no less than 275 nm; no less than 300 nm; 315 nm; no less than 325 nm; no less than 335 nm; no less than 345 nm; no less than 355 nm; no less than 365 nm; no less than 375 nm; no less than 385 nm; no less than 395 nm; or no less than 400 nm. In various instances, the UV wavelength may be no greater than 400 nm; no greater than 390 nm; no greater than 380 nm; no greater than 370 nm; no greater than 360 nm; no greater than 350 nm; no greater than 340 nm; no greater than 330 nm; no greater than 320 nm; no greater than 315 nm; no greater than 300 nm; no greater than 275 nm; or no greater than 254 nm.

The example method may comprise exposing the electrospun fibers to UV light for a total time of 1 hour to 8 hours. In various instances, the total time may be between 1 hour and 8 hours; 2 hours and 8 hours; 3 hours and 8 hours; 4 hours and 8 hours; 5 hours and 8 hours; 6 hours and 8 hours; 7 hours and 8 hours; 1 hour and 7 hours; 1 hour and 6 hours; 1 hour and 5 hours; 1 hour and 4 hours; 1 hour and 3 hours; or 1 hours and 2 hours. In various instances, the total time may be no less than 1 hour; no less than 2 hours; no less than 3 hours; no less than 4 hours; no less than 5 hours; no less than 6 hours; no less than 7 hours; or no less than 8 hours. In various instances, the total time may be no greater than 8 hours; no greater than 7 hours; no greater than 6 hours; no greater than 5 hours; no greater than 4 hours; no greater than 3 hours; no greater than 2 hours; or no greater than 1 hour.

After exposing the electrospun fibers to UV light, an elastomeric graft may be formed.

E. Method of Modifying Exemplary Grafts with Thioamide Pendant Groups

The exemplary method may comprise attaching pendant thioamide groups simultaneously with forming graft fibers and generating grafts, or attaching pendant thioamide groups after forming grafts.

Attaching pendant thioamide groups simultaneously with forming graft fibers and grafts may include combining a compound of formula (V), see below, with the polymer solution discussed above. The example method may comprise electrospinning the polymer solution combined with formula (V). After electrospinning, graft fibers integrated with formula (V) may be formed. The example method may include exposing the resultant electrospun graft fibers integrated with formula (V) to UV light. After exposing to UV light, an elastomeric graft with a plurality of pendant thioamide groups bonded to the graft via thioether bonds may be formed.

Attaching pendant thioamide groups after forming grafts may include combining a compound of formula (V), a solvent, a radical initiator, a weak organic base, and the elastomeric graft in a vessel. In some instances, the solvent may be ethanol or ethanol/acetone combination. In some instances, the radical initiator may be irgacure 2959 or may be ACVA (4,4′-azobis(4-cyanovaleric acid)). In some instances, the weak organic base may be triethylamine.

Formula (V) may be

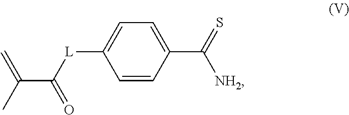

where L may be O or NH.

The example method may comprise exposing the vessel to heat or to UV light to form an elastomeric graft modified with a plurality of pendant thioamide groups bonded to the graft via thioether bonds.

The example may comprise exposing the reaction mixture to UV light between 254 nm and 400 nm at approximately room temperature. In various instances, the UV wavelength may be between 254 nm and 400 nm; 254 nm and 400 nm; 275 nm and 400 nm; 300 nm and 400 nm; 325 nm and 400; 350 nm and 400 nm; 375 nm and 400; 254 nm and 375 nm; 254 nm and 350 nm; 254 nm and 325 nm; 254 nm and 300 nm; or 254 nm and 275 nm. In various instances, the UV wavelength may be no less than 254 nm; no less than 275 nm; no less than 300 nm; 315 nm; no less than 325 nm; no less than 335 nm; no less than 345 nm; no less than 355 nm; no less than 365 nm; no less than 375 nm; no less than 385 nm; no less than 395 nm; or no less than 400 nm. In various instances, the UV wavelength may be no greater than 400 nm; no greater than 390 nm; no greater than 380 nm; no greater than 370 nm; no greater than 360 nm; no greater than 350 nm; no greater than 340 nm; no greater than 330 nm; no greater than 320 nm; no greater than 315 nm; no greater than 300 nm; no greater than 275 nm; or no greater than 254 nm.

The example may comprise heating the reaction mixture to a temperature between 30° C. and 70° C. In various instances, the temperature may be between 30° C. and 70° C.; 40° C. and 70° C.; 50° C. and 70° C.; 60° C. and 70° C.; 30° C. and 60° C.; 30° C. and 50° C.; or 30° C. and 40° C. In various instances the temperature may be no less than 30° C.; no less than 40° C.; no less than 50° C.; no less than 60° C.; or no less than 70° C. In various instances, the temperature may be no greater than 70° C.; no greater than 60° C.; no greater than 50° C.; no greater than 40° C.; or no greater than 30° C.

The example method may comprise reacting the mixture for a time period of 0.1 hours to 7 hours. In various instances, the time period may be between 0.1 hours and 7 hours; 0.1 hours and 6 hours; 0.1 hours and 5 hours; 0.1 hours and 4 hours; 0.1 hours and 3 hours; 0.1 hours and 2 hours; 0.1 hours and 1 hour; 1 hours and 7 hours; 2 hours and 7 hours; 3 hours and 7 hours; 4 hours and 7 hours; 5 hours and 7 hours; or 6 hours and 7 hours. In various instances, the time period may be no less than 0.1 hours; no less than 1 hour; no less than 2 hours; no less than 3 hours; no less than 4 hours; no less than 5 hours; no less than 6 hours; or no less than 7 hours. In various instances, the time period may be no greater than 7 hours; no greater than 6 hours; no greater than 5 hours; no greater than 4 hours; no greater than 3 hours; no greater than 2 hours; no greater than 1 hours; or no greater than 0.1 hours.

### IV. Articles of Manufacture

Exemplary grafts may have various sizes, configurations, and mechanical properties.

Mixtures of first polymers and second polymers may be electrospun and crosslinked to form exemplary grafts, which may be collected on a rotating mandrel. Exemplary grafts may be detached from the mandrel. Exemplary graft inner diameters may be controlled by a mandrel's diameter. Exemplary grafts may be fabricated from low, medium, or high average number molecular weight (Mn) first and second polymers. Exemplary grafts may be bent without kinks.

### A. Size of Graft

Exemplary grafts may have various sizes depending on intended use. Exemplary graft sizes were measured by FE-SEM imaging using a Hitachi S-4700. Exemplary graft average pore sizes and average fiber diameters may be adjusted by the weight ratios of first polymers to second polymers and the concentrations of the first polymers and second polymers. Graft wall thickness may be controlled by electrospinning time.

Exemplary grafts may have weight ratios of first polymers to second polymers between 1:1 and 1.4:1. In various instances, weight ratios of first polymers to second polymers may be between 1.00:1 and 1.40:1; 1.05:1 and 1.40:1; 1.10:1 and 1.40:1; 1.15:1 and 1.40:1; 1.20:1 and 1.40:1; 1.25:1 and 1.40:1; 1.30:1 and 1.40:1; 1.35:1 and 1.40:1; 1.00:1 and 1.35:1; 1.00:1 and 1.30:1; 1.00:1 and 1.25:1; 1.00:1 and 1.20:1; 1.00:1 and 1.15:1; 1.00:1 and 1.10:1; or 1.00:1 and 1.05:1. In various instances, weight ratios of first polymers to second polymers may be no less than 1.00:1; no less than 1.05:1; no less than 1.10:1; no less than 1.15:1; no less than 1.20:1; no less than 1.25:1; no less than 1.30:1; no less than 1.35:1; or no less than 1.40:1. In various instances, weight ratios of first polymers to second polymers may be no greater than 1.40:1; no greater than 1.35:1; no greater than 1.30:1; no greater than 1.25:1; no greater than 1.20:1; no greater than 1.15:1; no greater than 1.10:1; no greater than 1.05:1; or no greater than 1.00:1.

Exemplary grafts may have average fiber diameters between 0.5 μm and 10 μm. In various instances, average diameters are between 0.5 μm and 10 μm; 1 μm and 10 μm; 2 μm and 10 μm; 3 μm and 10 μm; 4 μm and 10 μm; 5 μm and 10 μm; 6 μm and 10 μm; 7 μm and 10 μm; 8 μm and 10 μm; 9 μm and 10 μm; 0.5 μm and 9 μm; 0.5 μm and 8 μm; 0.5 μm and 7 μm; 0.5 μm and 6 μm; 0.5 μm and 5 μm; 0.5 μm and 4 μm; 0.5 μm and 3 μm; 0.5 μm and 2 μm; or 0.5 μm and 1 μm. In various instances, exemplary grafts may have average diameters of no less than 0.5 μm; no less than 1 μm; no less than 2 μm; no less than 3 μm; no less than 4 μm; no less than 5 μm; no less than 6 μm; no less than 7 μm; no less than 8 μm; no less than 8 μm; or no less than 10 μm. In various instances, exemplary grafts may have average diameters of no greater than 10 μm; no greater than 9 μm; no greater than 8 μm; no greater than 7 μm; no greater than 6 μm; no greater than 5 μm; no greater than 4 μm; no greater than 3 μm; no greater than 2 μm; no greater than 1 μm; or no greater than 0.5 μm.

Exemplary grafts may have average pore sizes on the outer surface between 2 μm and 35 μm. In various instances, average pore sizes are between 2 μm and 35 μm; 5 μm and 35 μm; 10 μm and 35 μm; 15 μm and 35 μm; 20 μm and 35 μm; 25 μm and 35 μm; 30 μm and 35 μm; 2 μm and 30 μm; 2 μm and 25 μm; 2 μm and 20 μm; 2 μm and 15 μm; 2 μm and 10 μm; or 2 μm and 5 μm. In various instances, exemplary grafts may have average pore sizes of no less than 2 μm; no less than 5 μm; no less than 10 μm; no less than 15 μm; no less than 20 μm; no less than 25 μm; no less than 30 μm; or no less than 35 μm. In various instances, exemplary grafts may have average pore sizes of no greater than 35 μm; no greater than 30 μm; no greater than 25 μm; no greater than 20 μm; no greater than 15 μm; no greater than 10 μm; no greater than 5 μm; or no greater than 2 μm.

### B. Configuration of Grafts

Exemplary grafts may have various configurations depending on intended use. In some instances, exemplary grafts may comprise a tubular portion. In some instances, exemplary grafts may comprise a Y-shaped portion. In some instances, exemplary grafts may comprise a T-shaped portion. In some instances, exemplary grafts may comprise a planar portion. In some instances, exemplary grafts may comprise a spherical portion.

### C. H₂S Release

Pendant thioamides of exemplary grafts may be hydrolyzed or otherwise reacted to produce or release H₂S.

Exemplary grafts may release H₂S at an average rate of approximately 20 nM/day per mg graft to approximately 6,000 nM/day per mg graft. In various instances, an average release rate is between 20 nM/day per mg graft and 6,000 nM/day per mg graft; 100 nM/day per mg graft and 6,000 nM/day per mg graft; 500 nM/day per mg graft and 6,000 nM/day per mg graft; 1,000 nM/day per mg graft and 6,000 nM/day per mg graft; 1,500 nM/day per mg graft and 6,000 nM/day per mg graft; 2,000 nM/day per mg graft and 6,000 nM/day per mg graft; 2,500 nM/day per mg graft and 6,000 nM/day per mg graft; 3,000 nM/day per mg graft and 6,000 nM/day per mg graft; 3,500 nM/day per mg graft and 6,000 nM/day per mg graft; 4,000 nM/day per mg graft and 6,000 nM/day per mg graft; 4,500 nM/day per mg graft and 6,000 nM/day per mg graft; 5,000 nM/day per mg graft and 6,000 nM/day per mg graft; 5,500 nM/day per mg graft and 6,000 nM/day per mg graft; 20 nM/day per mg graft and 5,500 nM/day per mg graft; 20 nM/day per mg graft and 5,000 nM/day per mg graft; 20 nM/day per mg graft and 4,500 nM/day per mg graft; 20 nM/day per mg graft and 4,000 nM/day per mg graft; 20 nM/day per mg graft and 3,500 nM/day per mg graft; 20 nM/day per mg graft and 3,000 nM/day per mg graft; 20 nM/day per mg graft and 2,500 nM/day per mg graft; 20 nM/day per mg graft and 2,000 nM/day per mg graft; 20 nM/day per mg graft and 1,500 nM/day per mg graft; 20 nM/day per mg graft and 1,000 nM/day per mg graft; 20 nM/day per mg graft and 500 nM/day per mg graft; or 20 nM/day per mg graft and 100 nM/day per mg graft. In various instances, exemplary grafts may release H₂S at an average rate of no less than 20 nM/day per mg graft; no less than 100 nM/day per mg graft; no less than 500 nM/day per mg graft; no less than 1,000 nM/day per mg graft; no less than 1,500 nM/day per mg graft; no less than 2,000 nM/day per mg graft; no less than 2,500 nM/day per mg graft; no less than 3,000 nM/day per mg graft; no less than 3,500 nM/day per mg graft; no less than 4,000 nM/day per mg graft; no less than 4,500 nM/day per mg graft; no less than 5,000 nM/day per mg graft; no less than 5,500 nM/day per mg graft; or no less than 6,000 nM/day per mg graft. In various instances, exemplary grafts may release H₂S at an average rate of no greater than 6,000 nM/day per mg graft; no greater than 5,500 nM/day per mg graft; no greater than 5,000 nM/day per mg graft; no greater than 4,500 nM/day per mg graft; no greater than 4,000 nM/day per mg graft; no greater than 3,500 nM/day per mg graft; no greater than 3,000 nM/day per mg graft; no greater than 2,500 nM/day per mg graft; no greater than 2,000 nM/day per mg graft; no greater than 1,500 nM/day per mg graft; no greater than 1,000 nM/day per mg graft; no greater than 500 nM/day per mg graft; no greater than 100 nM/day per mg graft; or no greater than 20 nM/day per mg graft.

Exemplary grafts may release H₂S at no less than 20 nM/day per mg graft for a period of approximately 12 days to 24 days. In various instances, a period of release is between 12 days and 24 days; 13 days and 24 days; 14 days and 24 days; 15 days and 24 days; 15 days and 24 days; 16 days and 24 days; 17 days and 24 days; 18 days and 24 days; 19 days and 24 days; 20 days and 24 days; 21 days and 24 days; 22 days and 24 days; 23 days and 24 days; 12 days and 23 days; 12 days and 22 days; 12 days and 21 days; 12 days and 20 days; 12 days and 19 days; 12 days and 18 days; 12 days and 17 days; 12 days and 16 days; 12 days and 15 days; 12 days and 14 days; or 12 days and 13 days. In various instances, exemplary grafts may have a release period of no less than 12 days; no less than 13 days; no less than 14 days; no less than 15 days; no less than 16 days; no less than 17 days; no less than 18 days; no less than 19 days; no less than 20 days; no less than 21 days; no less than 22 days; no less than 23 days; or no less than 24 days. In various instances, exemplary grafts may have a release period of no greater than 24 days; no greater than 23 days; no greater than 22 days; no greater than 21 days; no greater than 20 days; no greater than 19 days; no greater than 18 days; no greater than 17 days; no greater than 16 days; no greater than 15 days; no greater than 14 days; no greater than 13 days; or no greater than 12 days.

### D. Elastic Modulus

Exemplary grafts may have an elastic modulus between 0.6 MPa and 6 MPa. In various instances, elastic modulus is between 0.6 MPa and 6 MPa; 1.2 MPa and 6 MPa; 1.8 MPa and 6 MPa; 2.4 MPa and 6 MPa; 3 MPa and 6 MPa; 3.6 MPa and 6 MPa; 4.2 MPa and 6 MPa; 4.8 MPa and 6 MPa; 5.4 MPa and 6 MPa; 0.6 MPa and 5.4 MPa; 0.6 MPa and 4.8 MPa; 0.6 MPa and 4.2 MPa; 0.6 MPa and 3.6 MPa; 0.6 MPa and 3 MPa; 0.6 MPa and 2.4 MPa; 0.6 MPa and 1.8 MPa; or 0.6 MPa and 1.2 MPa. In various instances, exemplary grafts may have an elastic modulus of no less than 0.6 MPa; no less than 1.2 MPa; no less than 1.8 MPa; no less than 2.4 MPa; no less than 3.0 MPa; no less than 3.6 MPa; no less than 4.2 MPa; no less than 4.8 MPa; no less than 5.4 MPa; or no less than 6 MPa. In various instances, exemplary grafts may have an elastic modulus of no greater than 6 MPa; no greater than 5.4 MPa; no greater than 4.8 MPa; no greater than 4.2 MPa; no greater than 3.6 MPa; no greater than 3 MPa; no greater than 2.4 MPa; no greater than 1.8 MPa; no greater than 1.2 MPa; or no greater than 0.6 MPa.

Elastic modulus may be determined using an MTS Acumen™ Electrodynamic Test System with a 3 kN loading cell (MTS Systems Corporation, MN, USA).

### E. Ultimate Tensile Strength

Exemplary grafts may have an ultimate tensile strength between 0.8 MPa and 6 MPa. In various instances, ultimate tensile strength is between 0.8 MPa and 6 MPa; 1 MPa and 6 MPa; 2 MPa and 6 MPa; 3 MPa and 6 MPa; 4 MPa and 6 MPa; 5 MPa and 6 MPa; 0.8 MPa and 5 MPa; 0.8 MPa and 4 MPa; 0.8 MPa and 3 MPa; or 0.8 MPa and 2 MPa. In various instances, exemplary grafts may have an ultimate tensile strength of no less than 0.8 MPa; no less than 1 MPa; no less than 2 MPa; no less than 3 MPa; no less than 4 MPa; no less than 5 MPa; or no less than 6 MPa. In various instances, exemplary grafts may have an ultimate tensile strength of no greater than 6 MPa; no greater than 5 MPa; no greater than 4 MPa; no greater than 3 MPa; no greater than 2 MPa; no greater than 1 MPa; or no greater than 0.8 MPa.

Ultimate tensile strength may be determined using an MTS Acumen™ Electrodynamic Test System with a 3 kN loading cell (MTS Systems Corporation, MN, USA).

### F. Fracture Strain

Exemplary grafts may have a fracture strain between 50% and 300%. In various instances, fracture strain is between 50% and 300%; 100% and 300%; 150% and 300%; 200% and 300%; 250% and 300%; 50% and 250%; 50% and 200%; 50% and 150%; or 50% and 100%. In various instances, exemplary grafts may have a fracture strain of no less than 50%; no less than 100%; no less than 150%; no less than 200%; no less than 250%; or no less than 300%. In various instances, exemplary grafts may have a fracture strain of no greater than 300%; no greater than 250%; no greater than 200%; no greater than 150%; no greater than 100%; or no greater than 50%.

Fracture strain may be determined using an MTS Acumen™ Electrodynamic Test System with a 3 kN loading cell (MTS Systems Corporation, MN, USA).

### G. Stress

At 80% strain, exemplary grafts may have a stress between 1000 kPa and 5000 kPa. In various instances, stress at 80% strain is between 1000 kPa and 5000 kPa; 2000 kPa and 5000 kPa; 3000 kPa and 5000 kPa; 4000 kPa and 5000 kPa; 1000 kPa and 4000 kPa; 1000 kPa and 3000 kPa; or 1000 kPa and 2000 kPa. In various instances, exemplary grafts may have a stress at 80% strain of no less than 1000 kPa; no less than 2000 kPa; no less than 3000 kPa; no less than 4000 kPa; or no less than 5000 kPa. In various instances, exemplary grafts may have a stress at 80% strain of no greater than 5000 kPa; no greater than 4000 kPa; no greater than 3000 kPa; no greater than 2000 kPa; or no greater than 1000 kPa.

Stress at 80% strain may be determined using an MTS Acumen™ Electrodynamic Test System with a 3 kN loading cell (MTS Systems Corporation, MN, USA).

### V. Experimental

Experimental embodiments were generated and tested. Experimental synthetic examples for polymers and grafts, polymer analysis, graft analysis, and H₂S release data are discussed below.

### A. Experimental Details

### 1. Synthesis of an Exemplary First Polymer

An exemplary first polymer was synthesized in two steps. In the first step, glycerol (3.684 g, 40.0 mmol), alkyne-serinol (1.852 g, 10.0 mmol), sebacic acid (10.112 g, 50.0 mmol), and 10 ml acetone (dried with 3 Å molecular sieves) were charged in a 100-ml three-neck round bottom flask. The mixture was purged with nitrogen gas for 30 min at room temperature. Then, the reaction temperature was increased to 125° C. to fully dissolve and mix the reactants for 2 h under nitrogen atmosphere. Next, polycondensation was conducted at 125° C. for 24 h with a gentle nitrogen purge through the reaction solution, followed by vacuum condensation (−29 inHg vacuum) for 5 h to yield a poly(glycerol-co-sebacate-co-alkyne-serinol) block with 20 mol. % alkyne pendants (PGSA₂₀).

In the second step, the PGSA₂₀ was further condensed with 50 mol. % PCL block to yield the resultant PCL₅₀-b-PGSA₂₀ copolyester, which is an exemplary first polymer. To this end, 80 kDa PCL (2.85 g, 25.0 mmol repeat units), sebacic acid (0.102 g, 0.50 mmol), and 10 ml 1,4-dioxane anhydrous were added to the PGSA₂₀ at room temperature. The reactants were purged with nitrogen gas for 30 min at room temperature. The reaction temperature was then increased to 120° C. to fully dissolve and mix the reactants for 4 h under nitrogen atmosphere with a magnetic stirring rate set at 600 rpm. Then, polycondensation was performed at 120° C. for 28 h with a gentle nitrogen purge through the reaction solution, followed by vacuum condensation (−29 inHg vacuum) for 7 h to yield the resultant exemplary first polymer, PCL₅₀-b-PGSA₂₀.

Before electrospinning, a purification procedure was used to remove a small amount of insoluble suspension in PCL₅₀-b-PGSA₂₀. After cooling down to room temperature, 100 ml acetone (dried with 3 Å molecular sieves) was added to fully dissolve the prepolymer at 50° C. overnight with a gentle magnetic stirring under nitrogen atmosphere. The PCL₅₀-b-PGSA₂₀ solution was cooled down to room temperature and then vacuum-filtered through a 0.80 μm PTFE membrane to remove insoluble suspension. The clear polymer solution was precipitated in 480 ml hexanes (40 ml per 50-ml centrifuge tube). The precipitates were agitated overnight on a tube rotator. The supernatant was decanted, followed by adding 40 ml of fresh hexanes to each tube and washing for another 24 h. The precipitates were then collected and dried at 60° C. in a vacuum oven (−29 inHg) for 24 h. The purified PCL₅₀-b-PGSA₂₀ in a sealed container was stored in a −20° C. fridge. The purified PCL₅₀-b-PGSA₂₀ was prepared for electrospinning.

### 2. Synthesis of an Exemplary Second Polymer

Thiomalic acid (40.0 mmol, 6.008 g), sebacic acid (40.0 mmol, 8.088 g), 1,8-ocanediol (80.0 mmol, 11.70 g), and 20 ml of acetone (dried with 3 Å molecular sieves) were charged in a 100-ml three-neck round bottom flask. The mixture was purged with nitrogen gas for 30 min at room temperature. The reaction was then heated to 140° C. The reactants were fully dissolved and mixed for 4 h under nitrogen atmosphere with a magnetic stirring rate set at approximately 450 rpm. Then, polycondensation remained at 140° C. for 20 h with a gentle nitrogen purge through the reaction solution, followed by vacuum condensation (−29 inHg) for 72 h with a magnetic stirring rate set at 300 rpm. After cooling down to room temperature, the resultant exemplary second polymer is poly(1,8-octanediol-co-sebacate-co-thiomalate) with 50 mol. % thiomalate (POST₅₀), which was collected and stored at −20° C.

3. Synthesis of Exemplary Thioamide Linker: 4-Carbamothioylphenyl methacrylate

4-Hydroxythiobenzamide (TBZ, 3.064 g, 20.0 mmol) was added to a dried round bottom flask along with 60 ml of anhydrous dichloromethane (DCM) and 30.0 mmol of triethylamine (TEA, 3.04 g, 4.2 ml). The flask was then set in an ice bath at 0-4° C. In a separate round bottom flask, 24.0 mmol of methacryloyl chloride (2.51 g, 2.3 ml) was mixed with 30 ml of anhydrous DCM that was also set in an ice bath. The chilled methacryloyl chloride/DCM solution was then added dropwise to the TBZ/DCM/TEA mixture via pipette. The reaction remained for 3 h. A clear orange solution was collected and washed with 100 ml of deionized water 3 times in a separating funnel. The resultant cloudy orange organic layer was then collected and dried with sodium sulfate for 10 minutes. The solution was then decanted into a round bottom flask and dried via rotary evaporation at 45° C. for 1.5 h to yield 4.77 g of a yellow-orange paste-like raw product. To further purify the exemplary thioamide linker, 450 ml of hexanes and ethyl acetate co-solvent (6:1 v/v) was added to wash the raw product. The mixture was gently shaken until there were no clumps of solid and only a light-yellow powder remained in the solution. The solid was then collected via vacuum filtration and dried in a vacuum chamber at room temperature for 24 h to obtain 1.01 g of purified exemplary thioamide linker, 22.8% yield.

### 4. Polymer Molecular Weight Analysis

The molecular weights of the PCL₅₀-b-PGSA₂₀ exemplary first polymer and POST₅₀ exemplary second polymers were analyzed using a size exclusion chromatography instrument (SHIMADZU HPLC Nexera Series) connected with a sample injection port, a degas system, a column oven, and three detectors. One precolumn (Shodex OHpak LB-G 6B, ID 8.00 mm×50.00 mm) and one column (Shodex OHpak LB-803, ID 8.00 mm×300.00 mm) were set as the stationary phase. N,N-dimethylformamide (DMF, HPLC grade) was used as the mobile phase. The detection system was equipped with a UV detector (SPD-40), a RI detector (RID-20A), and a multiple angle light scattering detector (WYATT, miniDAWN). ASTRA software (Version 8.1.2.1) was used for molecular weight analysis. Poly(styrene) standard (Mn/PDI, 30 kDa/1.02) at 5.00 mg/ml was used for calibration. The two polymer solutions were respectively prepared at 9.92 mg/ml and 9.97 mg/ml in DMF by incubating at 37° C. overnight and then filtered through a 0.45 μm syringe filter before test. DMF flow rate was set at 0.500 ml/min. The column oven temperature was set at 40° C., and the detection temperature was set at room temperature. Each injected sample solution was 20.0 μl.

### 5. Electrospinning to Fabricate an Exemplary Graft

To prepare PCL₅₀-b-PGSA₂₀ (PA) and POST₅₀ (PT) polymer solution for electrospinning, a polymer concentration at 29 wt./v % and a weight ratio at 1.2:1 was used for illustrative purposes. Specifically, 0.348 g of PA was weighed in a vial, followed by adding 0.48 ml of acetone and 0.72 ml of DCM to dissolve and yield a 29 wt./v % PA solution. In another vial, 0.290 g of PT was weighed and dissolved in 0.40 ml of acetone and 0.60 ml of DCM to yield a 29 wt./v % PT solution. The two polymer solutions were simultaneously prepared by vortexing at 500 rpm for 1 min and agitating at 30 rpm on a tube rotator for 24 h. Then, the two polymer solutions were combined, vortexed at 500 rpm for 1 min, and agitated at 30 rpm for 48 h. Next, 3.5 mg of irgacure 2959 (10 mol. % relative to alkyne groups in the PA) as a UV initiator was added to the mixture, followed by vortexing for 1 min and agitating at 30 rpm for 1.5 h. The polymer solution was then transferred to a 2-ml plastic syringe connected with a blunt needle (22 G, 3.8-cm length) for electrospinning. Other polymer solutions with different concentrations and polymer ratios were similarly prepared for electrospinning.

The solution-loaded syringe was set on an injection pump (SyringeONE 1000, New Era Pump Systems, Inc.). The needle was connected to a high-voltage power supply (GLASSMAN HIGH VOLTAGE, Inc., Series EL). A 1-mm stainless steel mandrel was used as a collector, which was grounded. The mandrel was coated with a thin layer of PEO (~10 μm thick) using a small painting brush and 1 wt. % of PEO (Mv: ~5M Da) in deionized water solution. The coated mandrel was dried at room temperature in a vacuum oven overnight. The injection rate, voltage, distance between the needle tip and the mandrel, and the mandrel rotating speed were set at 13.4 μl/min, 13.3 kV, 24 cm, and 50 rpm for electrospinning. The electrospun PAPT fibers were uniformly collected on each rotating mandrel. The thickness of the electrospun conduit was controlled at 200-250 μm, which was monitored by a digital micrometer (LS-7600 Series, Keyence).

After completion of the electrospinning, each end of the fiber-collected mandrel was inserted in a silicone disc center (6-mm diameter), and then put the mandrel in a glass tube to avoid touching the exemplary graft outer surface with the inner surface of the glass tube. The glass tube ends were covered by rubber septa and further sealed by Parafilm. The glass tube was then purged with nitrogen gas for ~10 min. Next, the glass tube was exposed under UV light at 365 nm (SPECTROLINE, Model ENF-280C) for 2 h and then rotated 180 degrees for another 2 h. The UV-cured PAPT conduit, which is an exemplary graft, was then immersed in 50 v/v % ethanol/deionized water overnight, and gently detached from the mandrel. The PAPT conduit was further washed by 75 v/v % ethanol/deionized water for 24 h and deionized water for 48 h, and freeze-dried.

6. Construction of Graft with Pendant Thioamides by Electrospinning

Using the PA/PT mixture at 29 wt./v % and a weight ratio at 1.2:1 as an example, the PA and PT polymer solutions were similarly prepared, combined, and agitated for 48 h as described above. Then, 6.4 mg of exemplary thioamide linker powder (1 wt. % relative to the mass of the PA and PT polymers) and 9.6 mg of irgacure 2959 were added to the PA/PT mixture, vortexed for 1 min, and agitated at 30 rpm for 1.5 h. The mixture was used for electrospinning as described above.

7. Construction of Graft with Pendant Thioamides by Conjugation

The electrospun PAPT conduit is an exemplary graft and was cut into 1.5-cm long conduits for conjugation reactions. Two conjugation methods to make PAPT conduits with pendant thioamides were attempted. In one method, a thermal initiator 4,4′-azobis(4-cyanopentanoic acid) (ACVA) was used to trigger the thiol-ene click reaction between the methacrylate group in the exemplary thioamide linker with the thiol group in the PAPT conduit (H₂S-PAPT_T). In another method, the UV initiator (irgacure 2959) was used to trigger the thiol-ene click reaction (H₂S-PAPT_UV).

To make H₂S-PAPT_UV, 22.4 mg exemplary thioamide linker (101 μmol) was dissolved in 3.4 ml ethanol (dried by 3 Å molecular sieves) to form a 30 mM thioamide linker solution in a 2-dram glass vial. Then, 0.113 g of the PAPT conduits bearing 50.6 μmol free thiol groups were immersed in the thioamide linker solution, which was incubated at 37° C. for 4 h. After cooling down to room temperature, 11.3 mg irgacure (50.6 μmol, equivalent to the thiol moles) was added to the solution. The vial was laid down and exposed under UV light at 365 nm (everBeam, 100 W) for 10 min. The UV light was turned off and the vial was gently swirled for about 2 min to cool down the solution temperature. The vial was exposed under the UV again for another 10 min for reaction. After the reaction, the unreacted thioamide linker solution was collected and the thioamide-conjugated PAPT conduits (H₂S-PAPT_UV), which are exemplary grafts with pendant thioamides, were washed with 5 ml ethanol (dried with 3 Å molecular sieves) three times, followed by adding 5 ml ethanol to further wash the H₂S-PAPT_UV conduits for ~24 h by agitation on a tube rotator. All washing solutions were combined with the unreacted thioamide linker solution and the unreacted thioamide linker was determined by UV-vis spectroscopy analysis using a thioamide linker standard curve. The washed H₂S-PAPT_UV conduits were then rinsed with 1 ml deionized water three times, freeze-dried, and stored at 4° C. fridge.

To make H₂S-PAPT_T conduit, the thioamide linker solution, its concentration, and the ratio of the thioamide linker to the thiol groups in the PAPT conduit were similar to that of the H₂S-PAPT_UV conduit. The UV initiator irgacure was replaced with ACVA (4,4′-azobis(4-cyanovaleric acid)). After adding ACVA to the incubated PAPT conduit/thioamide linker solution, the vial was further incubated at 50° C. for 5 h. The H₂S-PAPT_T conduits, which are exemplary grafts with pendant thioamides, were similarly washed and freeze-dried.

8. In Vitro H₂S Release Test of Exemplary Grafts with Pendant Thioamides

The as-prepared thioamide-conjugated PAPT conduits (H₂S-PAPT_E, and H₂S-PAPT_UV), which are exemplary grafts with pendant thioamides, were used for in vitro release test at 37° C. in pH 7.4 deionized water for 15 days. The H₂S-PAPT_E was sterilized by ethylene oxide (EtO) before the test to examine if the EtO sterilization would deactivate the thioamide or not. The H₂S-PAPT_T conduit was stopped at 6 days. The pH of the deionized water was adjusted by adding a suitable volume of 60 mM NaOH solution. Using H₂S-PAPT_UV conduit as an example, 5.0 ml of pH 7.4 deionized water was added to 44.0 mg of the conduits in a 2-dram vial. The vial was sealed by parafilm and incubated at 37° C. At each scheduled time point, the solution with released H₂S was collected and stored at −20° C. fridge. 5.0 ml of fresh pH 7.4 deionized water was added for the next time point. The H₂S concentration in each sample solution was determined by methylene blue assay kit (Sulfide Test, photometric, 0.020-1.50 mg/L (S²⁻), Spectroquant®, 1147790001, Millipore Sigma) according to the manufacturer's instruction.

### 9. Mechanical Property Test of Exemplary Grafts

1.5-cm long conduits were cut, and a 5-mm gap was marked on the center of each conduit. Uniaxial tensile test (n=6) was conducted to compare the mechanical properties with an elongation rate at 10 mm/min using MTS Acumen™ Electrodynamic Test System with a 3 kN loading cell (MTS Systems Corporation, MN, USA). For this MTS instrument, 21 data points were averaged to smooth the stress-strain curve for calculation of the Young's modulus (E), ultimate tensile strength (UTS), and fracture strain for each sample. The E was calculated from 1% strain to 20% strain.

10. SEM observation of Exemplary Grafts

2-mm conduit was cut circumferentially to observe the conduit cross-section. 4-mm conduit was cut longitudinally to observe inner and outer surfaces, respectively. All samples were coated with 7 nm gold layer using a sputter coater. FE-SEM imaging was performed on Hitachi S-4700.

### 11. Statistical Analysis

Statistical analysis was performed using one-way ANOVA analysis with a post-hoc Bonferroni's multiple comparison test. A p value<0.05 was considered significantly different. Data represented mean value±standard deviation (SD).

### B. Experimental Results

### 1. Syntheses of First and Second Polymers

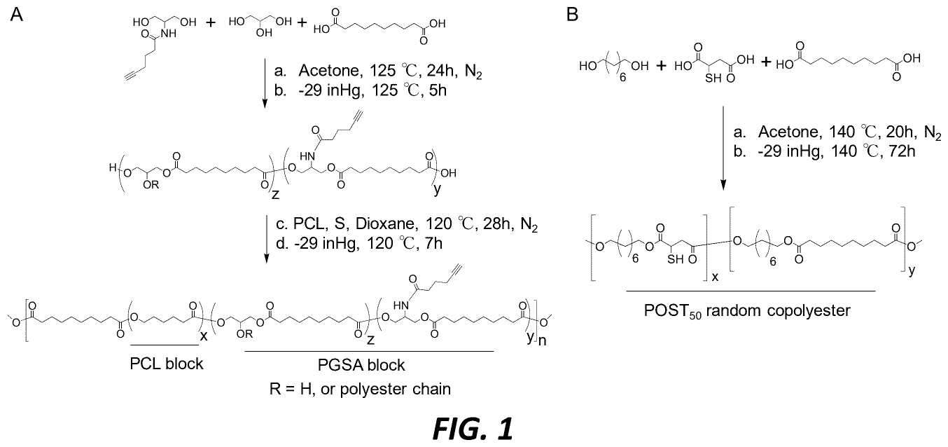

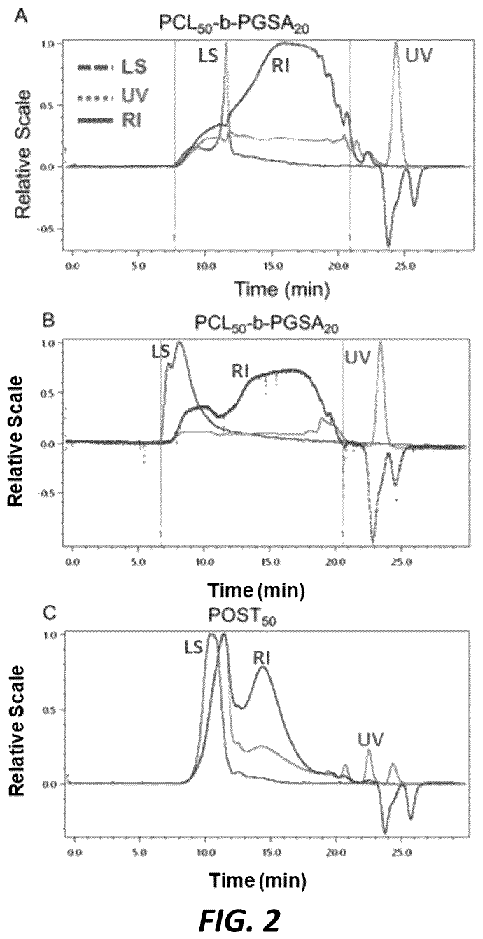

Exemplary first polymer was condensed from glycerol, alkyne-functionalized serinol, sebacic acid, and PCL block to yield poly(ε-caprolactone)-b-poly(glycerol-co-sebacate-co-alkyne-serinol) with 50 mol. % PCL block and 20 mol. % alkyne pendants (PCL₅₀-b-PGSA₂₀). Exemplary second polymer was condensed from 1,8-octanediol, sebacic acid, and thiomalic acid to yield poly(1,8-octanediol-co-sebacate-co-thiomalate) with 50 mol. % thiomalate (POST₅₀) (FIG. 1). FIG. 2 and Table 1 show gel permeation chromatography (GPC) data for PCL₅₀-b-PGSA₂₀ and POST₅₀. The Mn and PDI listed in Table 1 correspond to the current reactant quantities and the reaction conditions for these two prepolymers.

Table 1. Monomer quantities (mmol), number average molecular weight (Mn), and polydispersity (PDI) of PCL₅₀-b-PGSA₂₀ and POST₅₀. Poly(ε-caprolactone) (PCL), alkyne-functionalized serinol (AS), glycerol (G), sebacic acid(S), 1,8-octanediol (O), thiomalic acid (T). PCL quantities were based on its repeat units.

| Sample | | Mn (Da)/PDI |
|---|---|---|
| | PCL:AS:G:S, mmol | |
| PCL₅₀-b-PGSA₂₀ | 25.0:10.0:40.0:50.0 | 2020/22.5 |
| | 12.5:5.0:20.0:25.0 | 43,550/5.10 |
| | O:S:T, mmol | |
| POST₅₀ | 80.0:40.0:40.0 | 7180/24.2 |
| | 20.0:10.0:10.0 | 8990/4.15 |

### 2. Synthesis of Thioamide Linker

A thioamide linker was synthesized by reacting 4-hydroxythiobenzamide with methacryloyl chloride in the presence of triethylamine (TEA) in anhydrous dichloromethane (DCM) solvent at 0-4° C. for 3 hours under a nitrogen atmosphere.

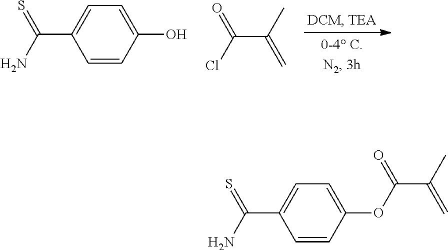

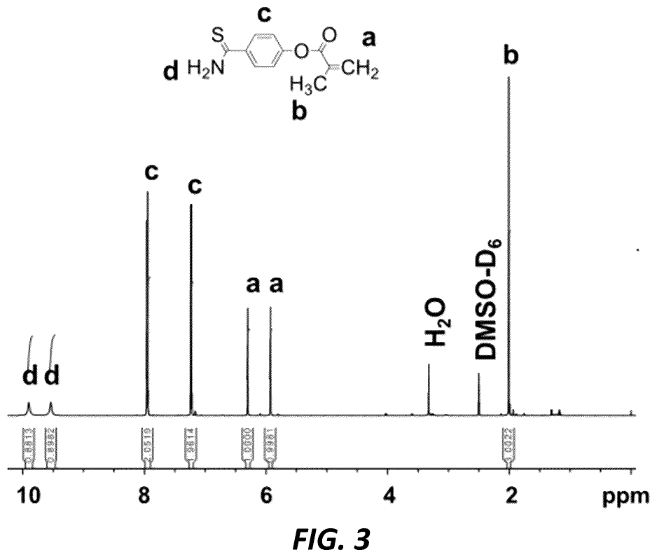

After purification, proton NMR analysis showed desired chemical shifts for different protons, including 2.0072 ppm for —CH₃ (H_{b}), 5.9257 ppm and 6.2996 ppm for ═CH₂ (Hₐ), 7.2221 ppm to 7.9613 ppm for the four aromatic protons (H_{c}), and 9.5364 ppm and 9.8991 ppm for —NH₂ (H_{d}) (FIG. 3). Integration area ratio of the Hₐ to H_{b} to H_{c} to H_{d} is equal to 2:3:4:1.8, which is close to theoretical ratio of 2:3:4:2.

### 3. Electrospinning and Photo-Crosslinking to Fabricate Fibrous Grafts

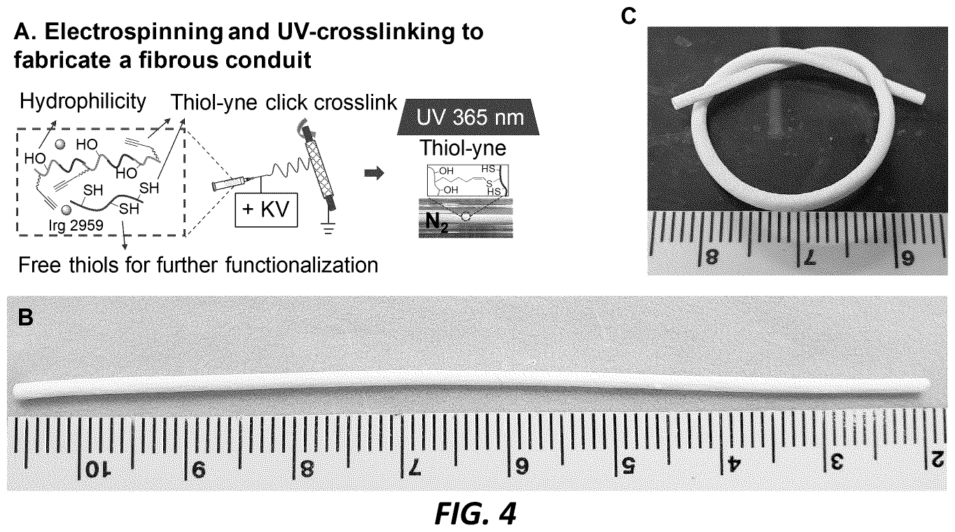

A mixture of the PCL₅₀-b-PGSA₂₀ (PA) and POST₅₀ (PT) polymers were electrospinnable and photocurable to fabricate elastic fibrous conduits (FIG. 4) with no need of a carrying polymer, thus easing fabrication of resorbable grafts for preclinical studies in different animal models.

Electrospinnability was examined by combining the high molecular weight PA and PT prepolymer pair (PAPT_H) and the low molecular weight PA and PT prepolymer pair (PAPT_L), respectively. With slightly different electrospinning conditions (Table 2), both of the combinations were electrospun and UV-crosslinked to form smooth elastic conduits (FIG. 4 (at B) and (at C)). The high molecular weight PA and PT mixture at 30-32 wt./v % in acetone and dichloromethane (2/3, v/v) was easier to establish physical interactions between the PA and PT polymer chains when compared to the low molecular weight PA and PT pair. Thus, a shorter mixing time and a slightly lower voltage was used for electrospinning to obtain a smooth PAPT conduit (FIG. 4 (at C)). FIG. 4 (at C) showed that the graft can be bent without a kink. The low molecular weight PA and PT pair at 29-30 wt. % needed a longer mixing time at 30 rpm on a tube rotator to establish suitable physical interactions between the PA and PT polymer chains for electrospinning to yield a smooth conduit (FIG. 4 (at B)). Compared to the high molecular weight PA and PT pair, the low molecular weight PA and PT pair was relatively easier to form a homogeneous mixture with a suitable viscosity for electrospinning. However, it required a longer mixing time and effort to avoid evaporation of the solvent. Under the electrospinning conditions, the PA polymer mixture alone and the PT polymer mixture alone of the tested concentration range formed conduits with severe fiber fusion and small pore sizes.

TABLE 2
Electrospinning conditions for the high molecular weight PA/PT pair
(PAPT_H) and low molecular weight PA/PT pair (PAPT_L).

| | | | Injection | | | Mandrel | |
|---|---|---|---|---|---|---|---|
| | Conc. | Mix. Time | Rate | Voltage | Distance | Rotating | Blunt |
| | wt./v % | h | μl/min | kV | cm | Rate, rpm | Needle |
| PAPT_H | 30-32% | 20-24 | 13.4 | 12.8 | 24 cm | 50 | 22 G |
| PAPT_L | 29-30% | 44-48 | 13.4 | 13.3 | 24 cm | 50 | 22 G |

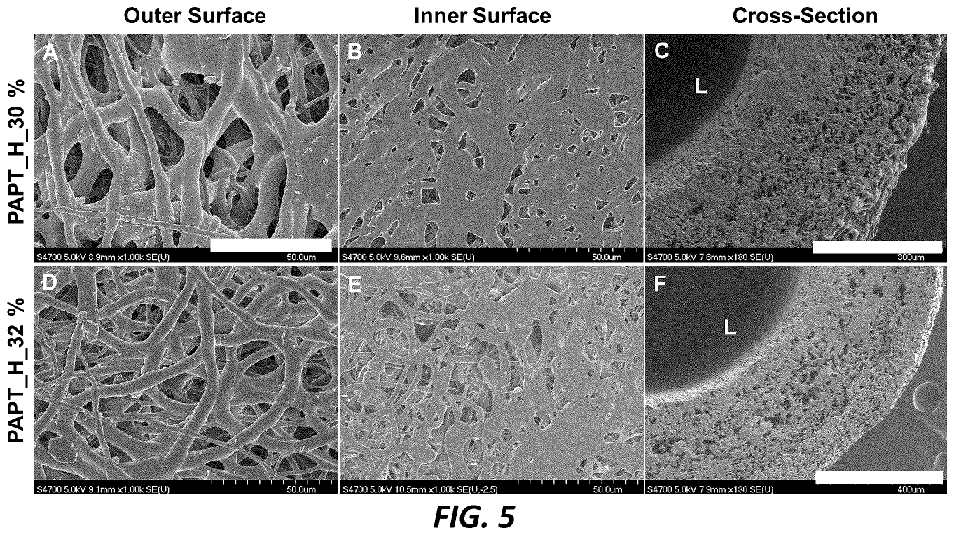

SEM observations showed that the electrospun conduits made from the high molecular weight PA and PT pair at 30 wt./v % and 32 wt./v % both possess pores between 5 and 30 μm on the outer surface (FIG. 5). Majority of the fiber diameter is 2-10 μm. The conduit wall thickness can be controlled by electrospinning time (FIG. 5 (at C) and (at F)).

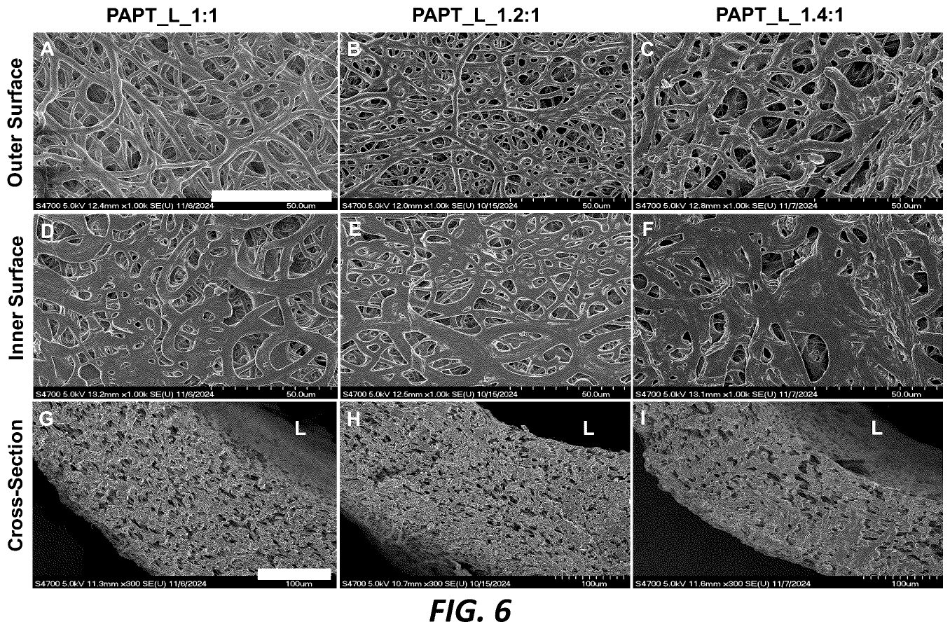

The low molecular weight PA and PT pair at 29 wt./v % was a more suitable concentration than 30 wt./v % mixture for electrospinning. The pore size and fiber diameters were adjusted by the PA-to-PT weight ratios. As the SEM images showed (FIG. 6), the PA-to-PT ratio at 1:1 (PAPT_L_1:1) yielded slightly thicker fibers (2-8 μm) and larger pore sizes (5-35 μm) on the outer surface compared to the PAPT_L_1.2:1 sample with fiber diameters at 1-5 μm and majority of pore sizes at 2-20 μm. Further increase of the PA-to-PT ratio to 1.4:1 led to fusion of the fibers and polymer aggregates. The fiber diameters of the PAPT_L_1:1 and PAPT_L_1.2:1 were smaller than those of the PAPT_H samples (FIG. 5 (at A) and (at D)).

Partial fusion of the fibers occurred on the overlap areas because of softness and viscous nature of the fibers before UV crosslinking. The fiber fusion was more severe on the inner surface which is in direct contact with the mandrel under the stress from outer layers (FIG. 5 and FIG. 6, inner surfaces).

Due to fiber fusion on the inner surface, the mandrel was coated with a thin layer of electrospun PEO fibers, which were then used to collect the electrospun PAPT fibers to increase the pore sizes on the inner surface. The electrospinning data evidenced that the PA and PT polymer pair with a wide range of molecular weights can be electrospun and UV-cured to construct elastic fibrous conduits. The polymer molecular weights, ratios, and concentrations were used to adjust the fiber diameters and the pore sizes of the electrospun conduits. The electrospinning conditions such as voltage, distance, and injection rate were further adjusted to impact fiber diameters and pore sizes.

### 4. Fabrication of Thioamide-Conjugated Grafts

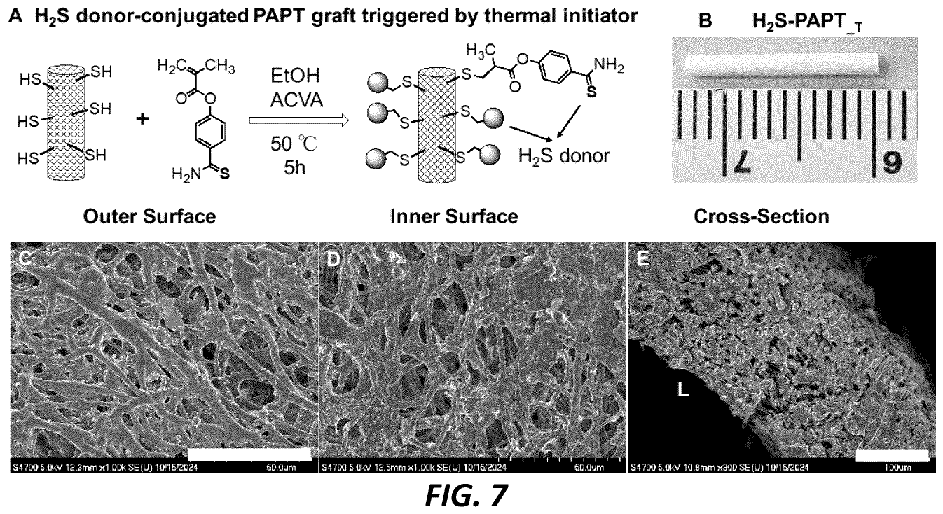

Three methods to conjugate the PAPT grafts with the thioamide linker were examined. PAPT grafts bear free thiol groups that were reacted with the methacrylate group of the thioamide linker through thiol-ene click chemistry that was triggered by a radical initiator (FIG. 7).

In the first conjugation method, the electrospun PAPT conduit was immersed in a solution of thioamide linker in ethanol, which was incubated at 37° C. for 4 hours. Then, a thermal radical initiator (ACVA) was added and incubated at 50° C. for 5 hours. SEM images showed that the fibers became partially fused under this conjugation condition, which reduced pore sizes (FIG. 7 (at C)). The fiber surfaces became rough with many micro- and nano-scale particles attaching to the fiber surfaces. The thermal initiator-triggered conjugation reactions mainly occurred on or close to the fiber surfaces.

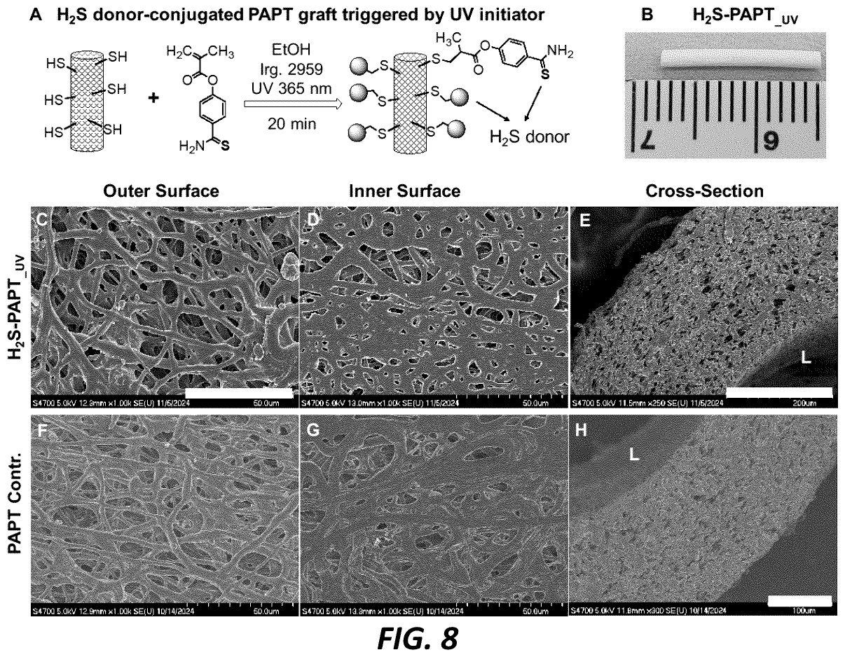

In the second conjugation method, the PAPT graft was similarly immersed in a solution of thioamide linker in ethanol for 4 hours, followed by adding a UV radical initiator, irgacure 2959. The resulting mixture was exposed to UV light at 365 nm for 20 minutes to trigger the conjugation reactions (FIG. 8 (at A)). Free thioamide linker residue was washed away, a slightly yellowish thioamide linked graft, or H₂S-releasing PAPT graft (H₂S-PAPT_UV), was obtained (FIG. 8 (at B)). SEM images showed little fiber fusion, pore collapse, or surface roughness by this conjugation method (FIG. 8 (at C), (at D), and (at E)). The fiber diameters and pore sizes may affect the mechanical properties, porosity, and degradation of the graft. The UV initiator-triggered conjugation appeared to be a suitable method to construct H₂S-releasing PAPT grafts as this method retained nearly the same microstructures of the graft when compared to the PAPT control graft (FIG. 8 (at F), (at G), and (at H)). Modifying thioamide loading on the graft may be used to modify or control the H₂S dosages and releasing kinetics.

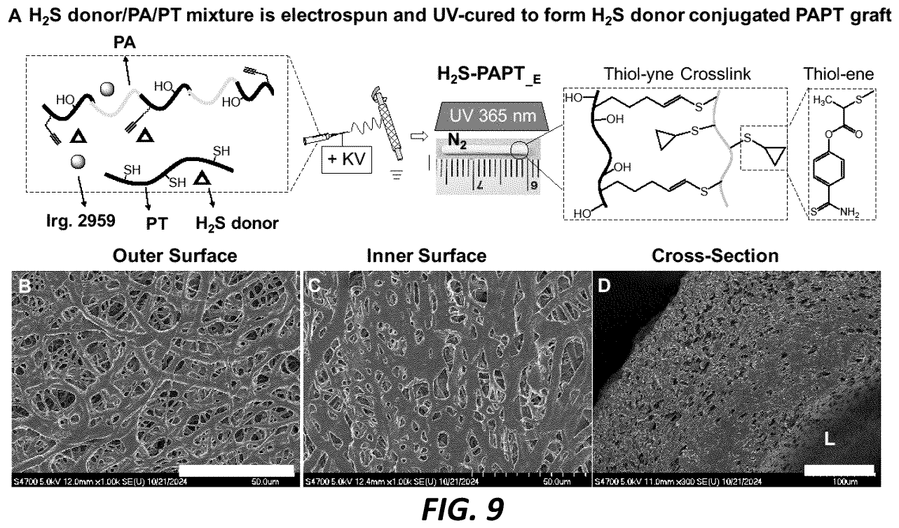

The third conjugation method used the thioamide/PA/PT mixture for electrospinning and UV-curing to make H₂S-releasing PAPT grafts (H₂S-PAPT__{E}) (FIG. 9). SEM observation showed smooth fiber surfaces, but a majority of the pore sizes on the outer surface were slightly reduced (FIG. 9 (at B)) compared to the PAPT control graft (FIG. 8 (at F)). Both the fiber surfaces and inner surfaces remained smooth (FIG. 9 (at B and C)). Thioamide linker (1 wt. %) was loaded in the PA/PT mixture for electrospinning and UV-curing to construct the H₂S-PAPT__{E} graft with a theoretical H₂S donor content at 0.045 μmol/mg graft for sustained release. This method needed to immerse the UV-cured graft in an ethanol/deionized water solution to detach it from the mandrel and was washed in ethanol/deionized water and deionized water to yield the resultant H₂S-PAPT__{E} graft. The immersion and washing processes may have hydrolyzed thioamides and decreased the H₂S donor loading.

According to the SEM observations of the above three methods (FIG. 7, FIG. 8, and FIG. 9), the UV initiator-triggered conjugation and the electrospinning method are suitable to fabricate H₂S-releasing grafts. PAPT grafts bore ~535 to 447 μmol free thiols/g PAPT when the PA-to-PT weight ratio was between 1:1 and 1.2:1, respectively. The electrospinning method loaded 1 wt. % thioamide linker in the PAPT graft (10 mol. % relative to the free thiols per g PAPT).

### 5. Sustained H₂S Release Test

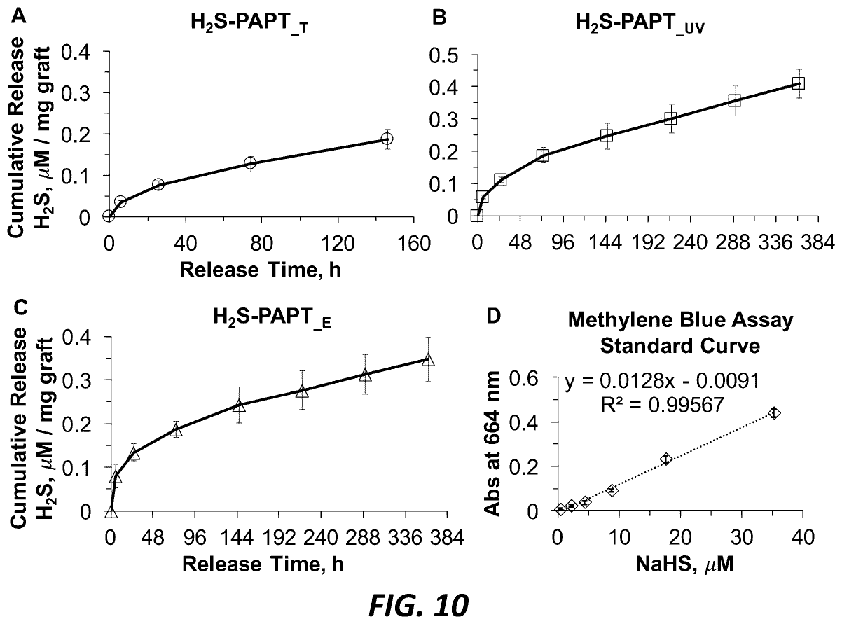

The H₂S releasing kinetics of the above three H₂S-releasing grafts were examined, namely H₂S-PAPT__{T}, H₂S-PAPT__{UV}, and H₂S-PAPT__{E}. An in vitro release test was performed by immersing these H₂S-PAPT grafts in deionized water (pH 7.4) at 37° C. for 15 days. The release test of the H₂S-PAPT__{T} sample was stopped at 6 days as this conduit showed partially fused fibers and pore structures (FIG. 7 (at C)). At scheduled time points, sample solutions containing the released H₂S were collected and the H₂S concentration was determined by methylene blue assay (FIG. 10).

Both the H₂S-PAPT__{UV} and the H₂S-PAPT__{E} grafts steadily released H₂S molecules for at least 15 days with a mild burst in the first 24 h. In addition, the H₂S-PAPT__{E} was sterilized by ethylene oxide (EO) and was used for the releasing test. A steady release of H₂S from the EO sterilized H₂S-PAPT__{E} indicated that the EO sterilization did not deactivate the conjugated H₂S donor. The H₂S-PAPT__{UV} graft was prepared by exposing under UV light at 365 nm (everBeam UV light, 100 W) for 20 min and also showed a steady release profile, which indicated that the UV exposure did not deactivate the conjugated H₂S donor.

The H₂S-PAPT__{UV} and the H₂S-PAPT__{E} grafts showed a steady H₂S release at an average rate of approximately 23 nM/Day/mg graft (16 μM/min/mg graft) for the H₂S-PAPT__{E} and approximately 27 nM/Day/mg graft (19 μM/min/mg graft) for the H₂S-PAPT__{UV} for at least 15 days, respectively.

### 6. Mechanical Property Tests

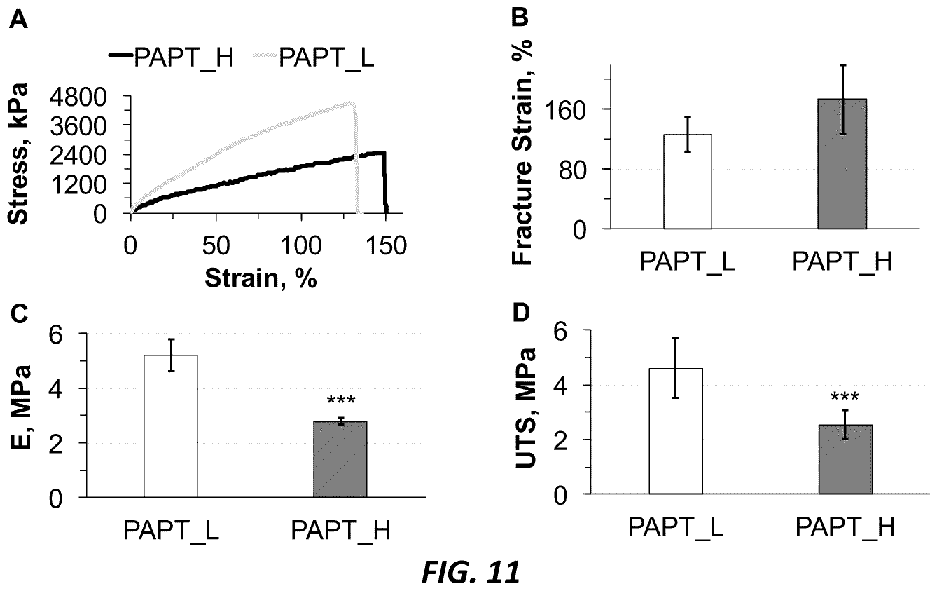

The mechanical properties of the PAPT grafts with and without conjugation of the thioamide were compared by uniaxial tensile tests. The PAPT grafts made from high molecular weight PA and PT pair (PAPT_H) and low molecular PA and PT pair (PAPT_L) (Table 1) were compared. The PAPT_H possessed a thicker fiber diameter and relatively larger pore sizes compared to the PAPT_L graft (FIG. 5 (at A) vs FIG. 8 (at F)). Both grafts showed nearly a linear elongation until break with a similar fracture strain (FIG. 11 (at A and B)). The PAPT_H graft showed a significantly lower elastic modulus and ultimate tensile strength compared to the PAPT_L graft (FIG. 11 (at C and at D)).

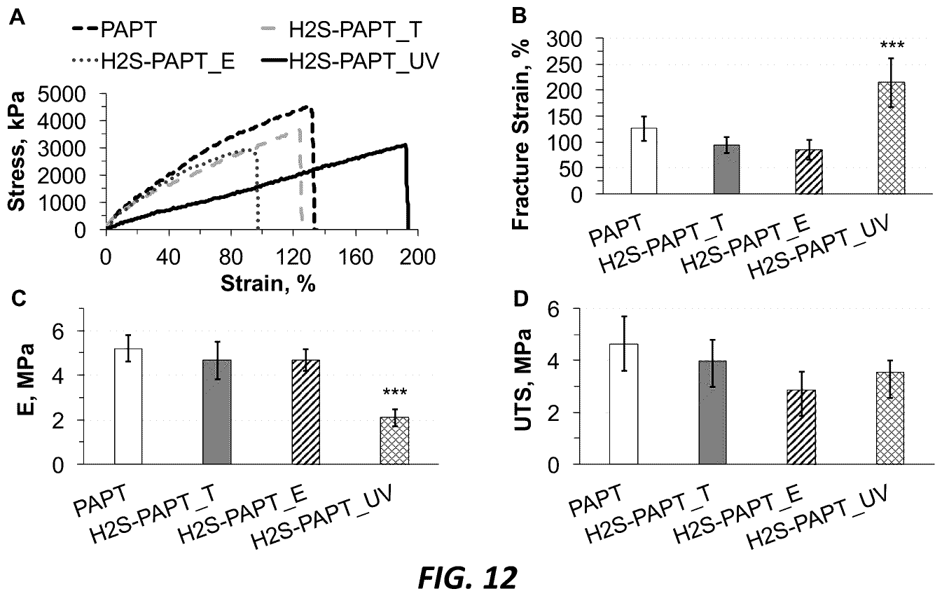

The mechanical properties of the PAPT graft were compared to the three types of H₂S-PAPT grafts (FIG. 12), which were all electrospun from a low molecular weight PA/PT mixture. The H₂S-PAPT__{UV} graft was significantly stretchable with an elastic modulus of 2.1±0.38 MPa, which was reduced by ~2.5 times that of the PAPT control (E: 5.2±0.59 MPa) (FIG. 12 (at C)). The tensile strength of the H₂S-PAPT__{UV} graft (UTS: 3.5±0.45 MPa) remained similar to the control conduit (4.6±1.1 MPa), FIG. 12 (at B). The other two H₂S-PAPT grafts (H₂S-PAPT__{T} and H₂S-PAPT__{E}) showed similar mechanical properties (E, UTS, and Fracture strain) to the PAPT control.

### 7. Studies in a Rat Abdominal Aorta Interposition Model

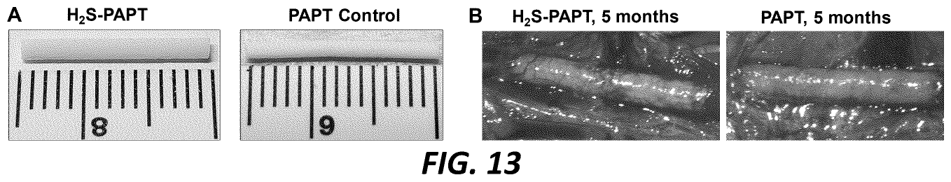

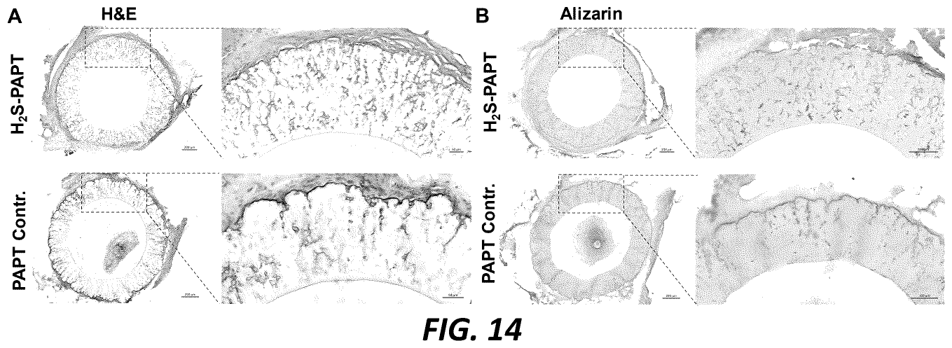

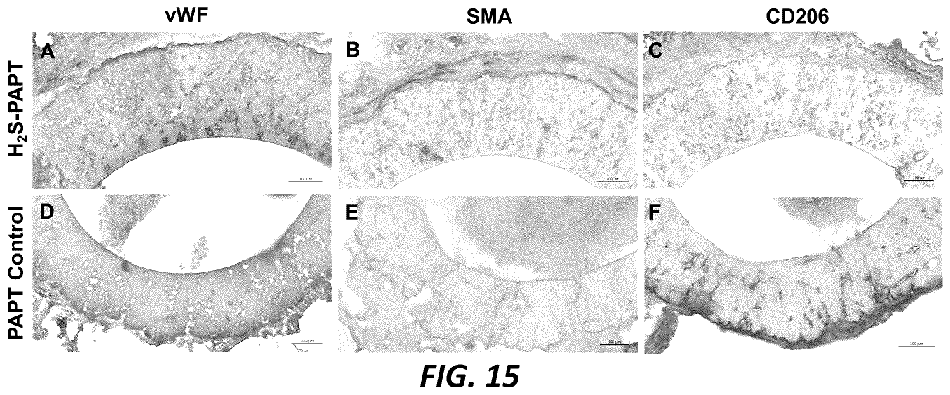

In this in vivo study, the thermal initiator-triggered conjugation method to construct the H₂S-PAPT conduit with fiber fusion and pore merge was used. Both the H₂S-PAPT graft and the PAPT control graft were implanted in the rat abdominal aorta interposition model over 5 months for a preliminary study. Histology and immunohistochemistry staining were used to detect the tissue organization, calcification, macrophage phenotype, endothelialization, and vascular smooth muscle cell distribution in these two types of remodeled grafts (FIG. 13, FIG. 14, and FIG. 15, n=4 per group).

The study revealed the following information. (1) The elastomer was biocompatible, biodegradable, and bioresorbable. (2) The H₂S-releasing PAPT graft was able to effectively inhibit thrombosis with a higher patency rate (75%) versus the control graft (0%) across the 5-months of remodeling. (3) The H₂S-releasing graft appeared to be more efficiently remodeled by the host when compared to the control graft. (4) The H₂S-releasing graft showed micro-vasculatures surrounding the adventitia surface, more smooth muscle cell growth in the adventitia and the graft wall, and more polarization of macrophages to M2 phenotype. (5) No calcification occurred in both the H₂S-releasing and the control grafts over 5 months, which indicated that the elastomer itself did not induce calcification and the sustainably released H₂S molecules might further inhibit potential calcification. These data qualitatively manifested that the released H₂S could stimulate angiogenesis and endothelialization, and modulate inflammatory responses to promote the graft regeneration with a higher patency rate. The H₂S-releasing graft was not fully remodeled over 5 months mainly because of too small pore size, low porosity, and limited cell infiltration and growth in the graft wall for remodeling.

### 8. Summary

The molecular weight of the first and second polymers, their ratio and concentration, and thioamide conjugation, among others, were used to control the graft parameters, such as fiber diameters, pore sizes, elasticity, mechanical properties, and degradation rate, as well as H₂S release kinetics.

1. An elastomeric graft comprising:
a backbone comprising a first polymer crosslinked with a second polymer, the first polymer comprising: 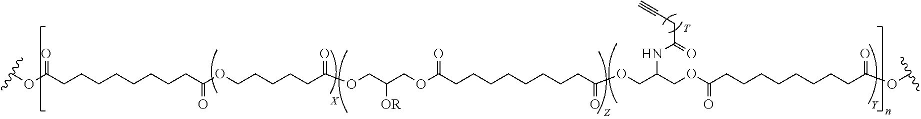
and
where a ratio of X to (Y+Z) is between 20:80 and 55:45;
where a ratio of Y to Z is between 10:90 and 25:75;
where T is an integer from 0 to 6;
where R is hydrogen or a polyester chain;
where n is an integer from 2 to 50; and
a second polymer comprising: 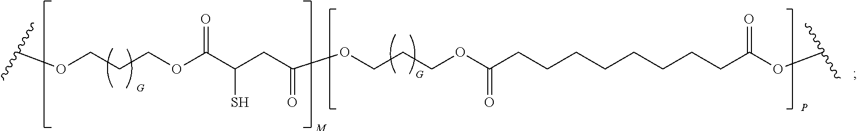
where a ratio of M to P is between 40:60 and 60:40; and
where G is an integer from 1 to 6.

2. The elastomeric graft according to claim 1, further comprising a plurality of thioamide pendant groups covalently attached to the backbone via a thioether bond.

3. The elastomeric graft according to claim 2, further comprising a plurality of thioamide pendant groups covalently attached to the backbone via a thioether bond,
wherein the thioamide is a primary thioamide or an aryl thioamide.

4. The elastomeric graft according to claim 1, wherein a number average molecular weight (Mₙ) of the first polymer is between 1,800 Daltons and 47,500 Daltons.

5. The elastomeric graft according to claim 1, wherein a polydispersity index (PDI) of the first polymer is between 5 and 68.

6. The elastomeric graft according to claim 1, wherein the first polymer and second polymer are crosslinked via vinyl thioether.

7. The elastomeric graft according to claim 1, wherein a number average molecular weight (Mn) of the second polymer is between 6400 Daltons and 9600 Daltons.

8. The elastomeric graft according to claim 1, wherein a polydispersity index of the second polymer is between 3 and 26.

9. The elastomeric graft according to claim 1, wherein an elastic modulus of the elastomeric graft is between 0.6 MPa and 6 MPa.

10. The elastomeric graft according to claim 1, wherein an ultimate tensile strength of the elastomeric graft is between 0.8 MPa and 6 MPa.

11. The elastomeric graft according to claim 1, wherein a fracture strain of the elastomeric graft is between 50 and 300%.

12. The elastomeric graft according to claim 1, wherein at 80% strain a stress is between 1000 kPa and 5000 kPa.

13. A method for preparing an elastomeric graft, the method comprising:
combining a compound of formula (I) with glycerol, sebacic acid, and a solvent in a first vessel, where formula (I) is: 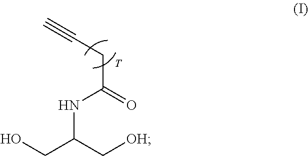
and
where T is an integer from 0 to 6; and
heating the first vessel to a temperature of 100° C. to 150° C.;
while heating, purging the first vessel with nitrogen (N₂) for a time period of 20 hours to 28 hours;
after purging with nitrogen and while heating, applying a vacuum to the first vessel for a time period of 3 hours to 7 hours; thereby generating a compound of formula (II): 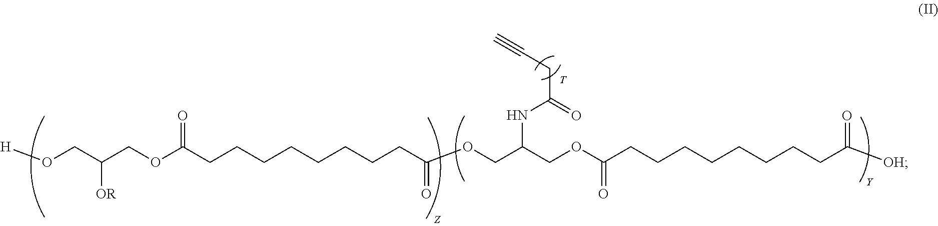
and
where a ratio of Y to Z is between 10:90 and 25:75; and
where R is hydrogen or a polyester chain;
combining the compound of formula (II) with poly(ε-caprolactone), sebacic acid, and a second solvent in a second vessel;
heating the second vessel to a temperature of 100° C. to 150° C.;
while heating, purging the second vessel with nitrogen for a time period of 20 hours to 28 hours;
after purging with nitrogen and while heating, applying a vacuum to the second vessel for a time period of 7 hours to 40 hours; thereby generating a first polymer;
combining a compound of formula (III), sebacic acid, a compound of formula (IV) and a third solvent in a third vessel, where formula (III) is: 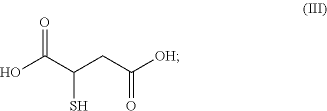
and
where formula (IV) is: 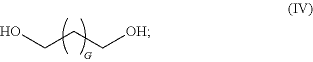
and
where G is an integer from 1 to 6;
heating the third vessel to a temperature of 100° C. to 150° C.;
while heating, purging the third vessel with nitrogen for a time period of 20 hours to 28 hours;
after purging with nitrogen and while heating, applying a vacuum to the third vessel for a time period of 70 hours to 80 hours; thereby generating a second polymer;
combining the first polymer, the second polymer, a fourth solvent, and a radical initiator to form a crosslinking mixture; and
electrospinning the crosslinking mixture to generate graft fiber.

14. The method of claim 13, further comprising:
combining a compound of formula (V), a solvent, a radical initiator, and the elastomeric graft in a fourth vessel, where formula (V) is: 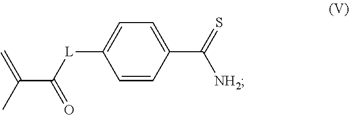
where L is O or NH; and
heating the fourth vessel or exposing the vessel to UV light thereby generating an elastomeric graft modified with a plurality of thioamide pendant groups covalently attached to the elastomeric graft via a thioether bond.

15. The method according to claim 13, further comprising exposing the elastomeric graft fiber to UV light thereby generating the elastomeric graft.

16. The method according to claim 15, wherein the UV light is applied for a time period of 1 hours to 8 hours.

17. The method according to claim 16, wherein the radical initiator comprises 2-hydroxy-4′-(2-hydroxyethoxy)-2-methylpropiophenone derivatives, azobisisobutyronitrile, azobisisobutyronitrile derivatives, benzoyl peroxide, benzoyl peroxide derivatives, 2,2-dimethoxy-2-phenylacetophenone, or 2,2-dimethoxy-2-phenylacetophenone derivatives.

18. The method according to claim 17, wherein a sum of the first polymer and the second polymer is between 25 wt % and 35 wt % by volume of the fourth solvent.

19. The method according to claim 18, wherein a wavelength of the UV light is between 254 nm and 400 nm.

20. The method according to claim 14, wherein the radical initiator is irgacure, or (4,4′-azobis(4-cyanovaleric acid)), or 2,2′-azobis(2,4-dimethylpentanenitrile).
